(19) United States
(12) Patent Application Publication
(10) Pub. No.: US 2025/0136605 A1
(43) Pub. Date: May 1, 2025

(54) IMPROVED METHODS FOR PREPARING CYTOTOXIC BENZODIAZEPINE DERIVATIVES

(71) Applicant: IMMUNOGEN, INC., Waltham, MA (US)

(72) Inventor: Andrew Brian Hague, Chelmsford, MA (US)

(21) Appl. No.: 18/683,361

(22) PCT Filed: Aug. 12, 2022
(86) PCT No.: PCT/US2022/040203

§ 371 Date: Feb. 13, 2024

Related U.S. Application Data
(60) Provisional application No. 63/232,757, filed on Aug. 13, 2021.

(51) Int. Cl. C07D 487/04; B01J 25/02; C07K 5/062

(52) U.S. Cl. CPC C07D 487/04; B01J 25/02; C07K 5/06026

(57) ABSTRACT

The invention provides novel improved methods for preparing indolinobenzodiazepine dimer compounds and their synthetic precursors.

### CROSS REFERENCE TO RELATED APPLICATIONS

This application claims priority to U.S. Provisional Application No. 63,232,757 filed on Aug. 13, 2021, the entire content of which is incorporated herein by reference.

### FIELD OF THE INVENTION

The present invention relates to improved methods for preparing cytotoxic indolinobenzodiazepine derivatives and precursors.

### BACKGROUND OF THE INVENTION

It has been shown that cell-binding agent conjugates of indolinobenzodiazepine dimers that have one imine functionality and one amine functionality display a much higher therapeutic index (ratio of maximum tolerated dose to minimum effective dose) in vivo compared to previously disclosed benzodiazepine derivatives having two imine functionalities. See, for example, WO 2012/128868. The previously disclosed methods for making the indolinobenzodiazepine dimers with one imine functionality and one amine functionality have various drawbacks and problems. For instance, one previously disclosed method involves partial reduction of indolinobenzodiazepine dimers having two imine functionalities. The partial reduction step generally leads to the formation of fully reduced by-product and unreacted starting material, which requires cumbersome purification step and results in low yield. Other problems in the previously disclosed methods may be caused by impurities generated during the processes of preparing the precursors, which are hard to remove and, as a result, will contaminate the precursors and eventually lead to main impurities in the final desired indolinobenzodiazepine dimer products. Additional problems associated with the previous methods include high costs of solvent and reagents, low stability of the products, long reaction time, etc.

Thus, there is a continued need to develop improved methods for preparing the indolinobenzodiazepine dimers that are more efficient and suitable for large scale manufacturing process.

### SUMMARY OF THE INVENTION

The present invention provides new methods for preparing indolinobenzodiazepine dimer compounds and synthetic precursors thereof.

In one aspect, the present invention relates to a method of preparing a compound of formula (III):

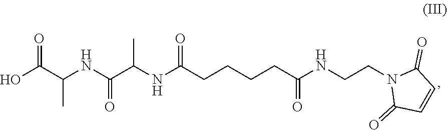 [SMILES: CC(NC(=O)C(C)NC(=O)CCCCC(=O)NCCN1C(=O)C=CC1=O)C(=O)O]

comprising the steps of
(i) reacting a compound of formula (I):

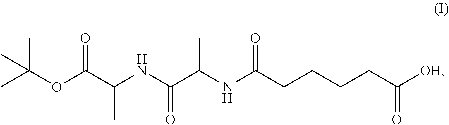 [SMILES: CC(NC(=O)CCCCC(=O)O)C(=O)NC(C)C(=O)OC(C)(C)C]

with a bisulfate salt (H₂SO₄ salt) of a compound of formula (b):

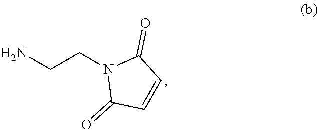

to form a compound of formula (II):

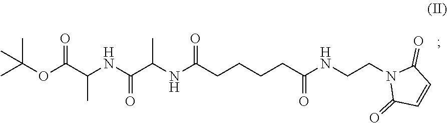 [SMILES: CC(NC(=O)CCCCC(=O)NCCN1C(=O)C=CC1=O)C(=O)NC(C)C(=O)OC(C)(C)C]

(ii) reacting the compound of formula (II) with a carboxylic acid deprotecting agent to form the compound of formula (III).

In another aspect, the present invention relates to a method of preparing a compound of formula (Va):

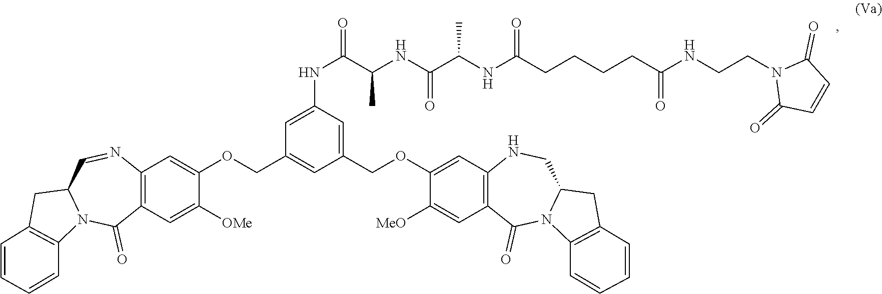 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O]

comprising the step of reacting a compound formula (IIIa)

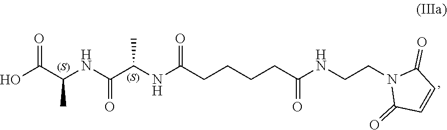

with a compound of formula (IVa):

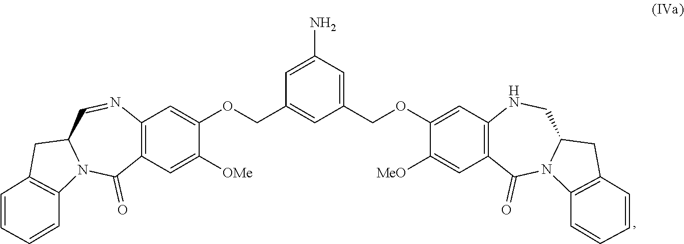 [SMILES: COc1cc2c(cc1OCc1cc(N)cc(COc3cc4c(cc3OC)C(=O)N3c5ccccc5C[C@H]3CN4)c1)N=C[C@@H]1Cc3ccccc3N1C2=O]

in the presence of an activating agent, in methanol to form the compound of formula (Va), wherein the activating agent is HATU.

In another aspect, the present invention provides a method of preparing a compound of formula (Va):

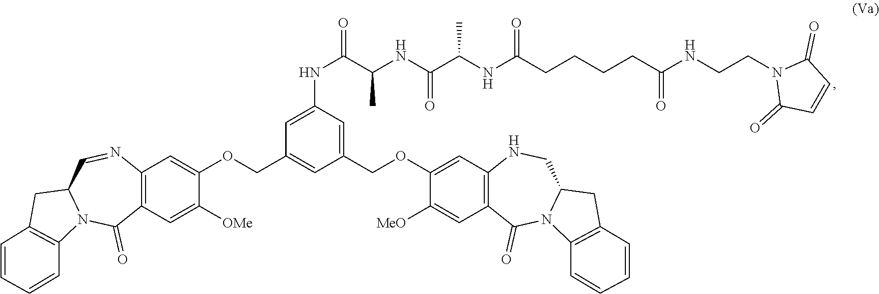 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O]

comprising the steps of:
(i) reacting a compound of formula (Ia):

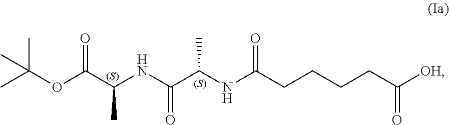

with a bisulfate salt (H₂SO₄ salt) of a compound of formula (b):

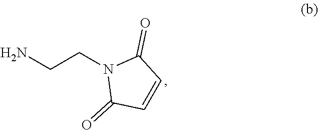

to form a compound of formula (IIa):

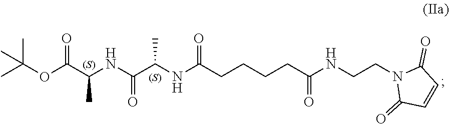

(ii) reacting the compound of formula (IIa) with a carboxylic acid deprotecting agent to form a compound of formula (IIIa):

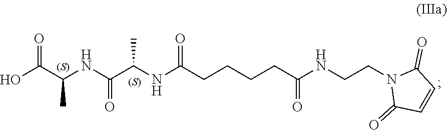

and
(iii) reacting the compound of formula (IIIa) with a compound of formula (IVa):

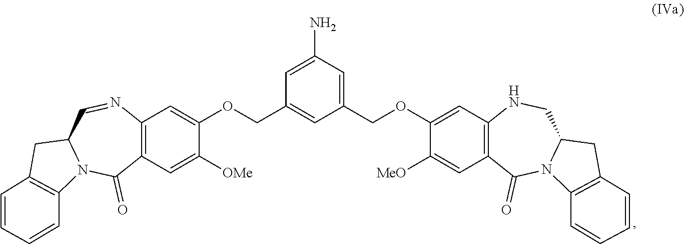 [SMILES: COc1cc2c(cc1OCc1cc(N)cc(COc3cc4c(cc3OC)C(=O)N3c5ccccc5C[C@H]3CN4)c1)N=C[C@@H]1Cc3ccccc3N1C2=O]

to form the compound of formula (Va).

In yet another aspect, the present invention provides a method of preparing a compound of formula (VIIa):

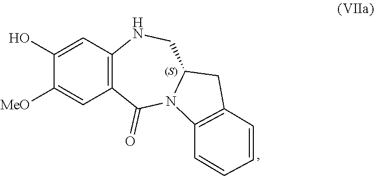

comprising the step of reacting a compound of formula (VIa):

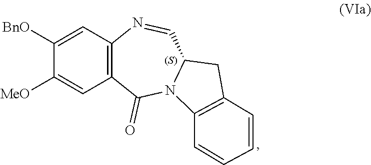

with H₂ in the presence of a Raney nickel catalyst.

Also provided herein is a method of preparing a compound of formula (VIIIa):

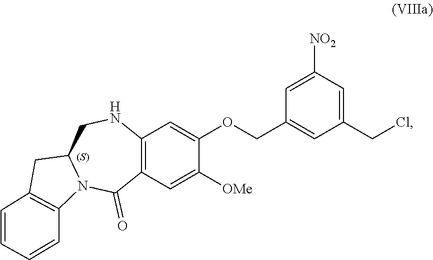

comprising the step of reacting a compound of formula (VIIa):

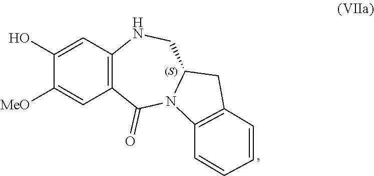

with a compound of formula (c):

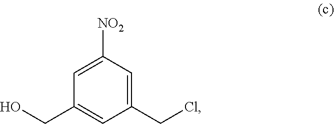 [SMILES: O=[N+]([O-])c1cc(CO)cc(CCl)c1]

to form the compound of formula (VIIIa), wherein the reaction is carried out in the presence of 1,2-bis(diphenylphosphino)ethane (DPPE) and 1,1′-(azodicarbonyl)dipiperidine (ADDP).

### DETAILED DESCRIPTION OF THE INVENTION

Reference will now be made in detail to certain embodiments of the invention, examples

of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention.

It should be understood that any of the embodiments described herein can be combined with one or more other embodiments of the invention, unless explicitly disclaimed or improper. Combination of embodiments are not limited to those specific combinations claimed via the multiple dependent claims.

### Definitions

“Alkyl’ as used herein refers to a saturated linear or branched monovalent hydrocarbon radical. In preferred embodiments, a straight chain or branched chain alkyl has thirty or fewer carbon atoms (e.g., C₁-C₃₀ for straight chain alkyl group and C₃-C₃₀ for branched alkyl), and more preferably twenty or fewer carbon atoms. Even more preferably, the straight chain or branched chain alkyl has ten or fewer carbon atoms (i.e., C₁-C₁₀ for straight chain alkyl group and C₃-C₁₀ for branched alkyl). In other embodiments, the straight chain or branched chain alkyl has six or fewer carbon atoms (i.e., C₁-C₆ for straight chain alky group or C₃-C₆ for branched chain alkyl). Examples of alkyl include, but are not limited to, methyl, ethyl, 1-propyl, 2-propyl, 1-butyl, 2-methyl-1-propyl, —CH₂CH(CH₃)₂), 2-butyl, 2-methyl-2-propyl, 1-pentyl, 2-pentyl 3-pentyl, 2-methyl-2-butyl, 3-methyl-2-butyl, 3-methyl-1-butyl, 2-methyl-1-butyl, 1-hexyl), 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, and the like. Moreover, the term “alkyl” as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. As used herein, (Cₓ-Cₓₓ)alkyl or Cₓ₋ₓₓalky means a linear or branched alkyl having x-xx carbon atoms.

As used herein, an “activated ester” refers to an ester group that is readily displaced by a hydroxyl or an amine group. Exemplary activated esters include, but are not limited to N-hydroxysuccinimide ester, nitrophenyl (e.g., 2 or 4-nitrophenyl) ester, dinitrophenyl (e.g., 2,4-dinitrophenyl) ester, sulfo-tetraflurophenyl (e.g., 4-sulfo-2,3,5,6-tetrafluorophenyl) ester, pentafluorophenyl ester, nitropyridyl (e.g., 4-nitropyridyl) ester, trifluoroacetate, and acetate.

The term “compound” is intended to include compounds for which a structure or formula or any derivative thereof has been disclosed in the present invention or a structure or formula or any derivative thereof that has been incorporated by reference. The term also includes, stereoisomers, geometric isomers, or tautomers. The specific recitation of “stereoisomers,” “geometric isomers,” “tautomers,” “salt” in certain aspects of the invention described in this application shall not be interpreted as an intended omission of these forms in other aspects of the invention where the term “compound” is used without recitation of these other forms.

The term “precursor” of a given group refers to any group which may lead to that group by any deprotection, a chemical modification, or a coupling reaction.

The term “chiral” refers to molecules that are non-superimposable on their mirror image partner, while the term “achiral” refers to molecules which are superimposable on their mirror image partner.

The term “stereoisomer” refers to compounds which have identical chemical constitution and connectivity, but different orientations of their atoms in space that cannot be interconverted by rotation about single bonds.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as crystallization, electrophoresis, and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds,” John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The term “protecting group” or “protecting moiety” refers to a substituent that is commonly employed to block or protect a particular functionality while reacting other functional groups on the compound, a derivative thereof, or a conjugate thereof.

An “carboxylic acid protecting group” is a substituent attached to the carbonyl group or the alcohol group of the carboxylic acid functionality that blocks or protects the carboxylic acid functionality in the compound. Such groups are well known in the art (see for example, P. Wuts and T. Greene, 2007, Protective Groups in Organic Synthesis, Chapter 5, J. Wiley & Sons, NJ). Suitable carboxylic acid protecting group include, but are not limited to, alkyl ester (e.g., methyl ester or tert-butyl ester), benzyl ester, thioester (e.g., tert-butyl thioester), silyl ester (e.g., trimethylsilyl ester), 9-fluorenylmehtyl ester, (2-trimethylsilyl)ethoxymethyl ester, 2-(trimethylsilyl)ethyl ester, diphenylmethyl ester or oxazoline. In certain embodiments, the carboxylic acid protecting group is methyl ester, tert-butyl ester, benzyl ester or trimethylsilyl ester. In certain embodiments, the carboxylic acid protecting group is tert-butyl ester.

As used herein, “carboxylic acid deprotecting agent” refers a reagent that is capable of cleaving a carboxylic acid protecting group to generate the free carboxylic acid. Such reagents are well known in the art (see for example P. Wuts and T. Greene, 2007, Protective Groups in Organic Synthesis, Chapter 5, J. Wiley & Sons, NJ) and depend on the carboxylic acid protecting group used. For example, when the carboxylic acid protecting group is tert-butyl ester, it can be cleaved with an acid. In certain embodiment, the carboxylic acid deprotecting agent is trifluoroacetic acid.

As used herein, “alcohol activating agent” refers a reagent that increases the reactivity of a hydroxyl group thereby making the hydroxyl group a better leaving group. Examples of such alcohol activating agents include p-toluenesulfonyl chloride, thionyl chloride, triflic anhydride, mesyl chloride, mesyl anhydride, triphenylphosphine, acyl chloride, 4-dimethylaminopyridine, and others. In certain embodiments, the alcohol activating agent is thionyl chloride. In certain embodiment, the alcohol activating agent is triphenylphosphine.

The phrase “salt” as used herein, refers to an organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A salt may involve the inclusion of another molecule such as an acetate ion, a succinate ion or other counter ion. The counter ion may be any organic or inorganic moiety that stabilizes the charge on the parent compound. Furthermore, a salt may have more than one charged atom in its structure. Instances where multiple charged atoms are part of the salt can have multiple counter ions. Hence, a salt can have one or more charged atoms and/or one or more counter ion.

If the compound of the invention is a base, the desired salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, hippuric acid, a pyranosidyl acid, such as glucuronic acid or galacturonic acid, an alpha hydroxy acid, such as citric acid, tartaric acid, or (+)-O,O′-di-p-toluoyl-D-tartaric acid (DTTA), an amino acid, such as aspartic acid or glutamic acid, an aromatic acid, such as benzoic acid or cinnamic acid, a sulfonic acid, such as benzenesulfonic acid, p-toluenesulfonic acid, camphorsulfonic acid or ethanesulfonic acid, or the like.

If the compound of the invention is an acid, the desired salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In certain embodiments, the salt is a pharmaceutically acceptable salt. The phrase “pharmaceutically acceptable” indicates that the substance or composition must be compatible chemically and/or toxicologically, with the other ingredients comprising a formulation, and/or the mammal being treated therewith.

As used herein, the term “scavenger” refers to a chemical substance added to a mixture in order to remove or de-activate excess reagents, catalysts, impurities and unwanted reaction products, for example oxygen, to make sure that they will not cause any unfavorable reactions. Scavengers include polymeric scavengers, radical scavengers, inorganic oxygen scavengers, etc. Metal scavengers are functionalized silica gels designed to react and bind excess metal complexes. Silica-based metal scavengers have been proven to be the purification method of choice over all other methods and several companies from various industries use them. With the silica matrix advantages over polymers (no swelling, more general solvent compatibility, higher mechanical and thermal stabilities, applications easily scalables and products available in different formats, i.e. SPE, flash cartridges, bulk, etc.) silica metal scavengers are the solution for metal removal without contamination of drug candidates. Examples of silica-based metal scavengers include Deloxan® MP Metal Scavengers (Thiol-Functionalized Polysiloxane), SiliCycle SiliaMetS® Metal Scavenger (Triamine), SiliaMetS® Thiol (Si-Thiol) Metal Scavenger, SiliCycle™ SiliaMetS™ Metal Scavengers Thiol (SH), Silicathiol (iMoLbox-LMat-NH01), etc.

### METHODS OF THE PRESENT INVENTION

The present invention provides novel synthetic methods for preparing indolinobenzodiazepine dimer compounds and precursors.

In a first embodiment, the present invention provides a method of preparing a compound of formula (III):

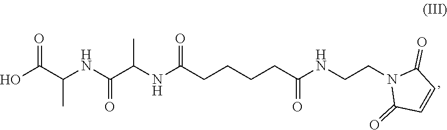 [SMILES: CC(NC(=O)C(C)NC(=O)CCCCC(=O)NCCN1C(=O)C=CC1=O)C(=O)O]

comprising the steps of
(i) reacting a compound of formula (I):

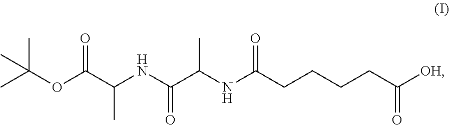 [SMILES: CC(NC(=O)CCCCC(=O)O)C(=O)NC(C)C(=O)OC(C)(C)C]

with a bisulfate salt (H₂SO₄ salt) of a compound of formula (b):

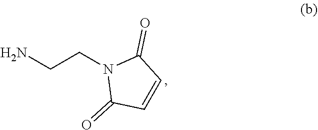

to form a compound of formula (II):

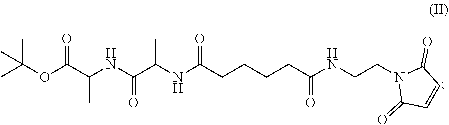 [SMILES: CC(NC(=O)CCCCC(=O)NCCN1C(=O)C=CC1=O)C(=O)NC(C)C(=O)OC(C)(C)C]

(ii) reacting the compound of formula (II) with a carboxylic acid deprotecting agent to form the compound of formula (III).

In a 1^{st} specific embodiment, the present invention provides a method of preparing a method of preparing a compound of formula (IIIa)

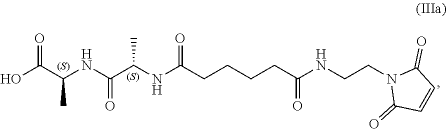

and the method comprises the steps of:
(i) reacting a compound of formula (Ia):

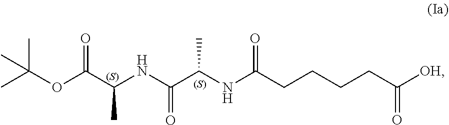

with the bisulfate salt (H₂SO₄ salt) of the compound of formula (b) to form a compound of formula (IIa):

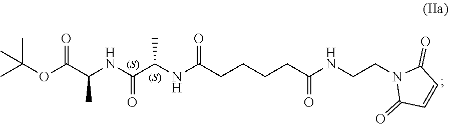

(ii) reacting the compound of formula (IIa) with a carboxylic acid deprotecting agent to form the compound of formula (IIIa).

In the previously disclosed method (e.g., WO 2020/102053), the compound of formula (IIIa) was prepared by reacting the compound of formula (Ia) with the TFA or HCl salt of the compound of formula (b) to obtain the compound of formula (IIa), followed by the next step Boc-deprotection reaction. Under the previously disclosed reaction conditions, the TFA salt of the compound of formula (b) can cause the formation of the trifluoroacetamide impurity Compound 1 depicted below, which is hard to remove from the reaction mixture and therefore contaminates the compound of formula (IIa). The use of the HCl salt of the compound of formula (b) may be limited by the potential reactivity of HCl with the maleimide group in the compound of formula (b), leading to undesired side-products.

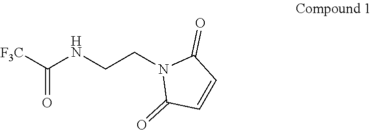 [SMILES: O=C1C=CC(=O)N1CCNC(=O)C(F)(F)F]

The present invention has found that the use of the bisulfate salt of the compound of formula (b) can avoid the formation of the trifluoroacetamide impurity and improves the efficiency and cost of the process, making it more suitable for large scale production.

In certain embodiments, the reaction between the compound of formula (Ia) and the bisulfate salt of the compound of formula (b) in the presence of an activating agent (e.g., T3P), which yields water-soluble by-products that can be easily removed from the reaction mixture by washing with water and an aqueous basic solution (e.g., NaHCO₃ solution). The unreacted compound of formula (Ia) can be eliminated as well by the aqueous wash. The resultant product (the compound of formula (IIa)) in the residual organic solvent after the aqueous wash can be directly carried over to the next Boc de-protection reaction without further purification (e.g., crystallization) that is usually needed in the conventional method.

When the compound of formula (IIa) prepared by the present methods is subjected to the next step of Boc-deprotection reaction, the resulting product (i.e., the compound of formula (IIIa)) can be formed with high purity and free of the diacid impurity Compound 2 depicted below, which is the Boc-deprotected by-product of unreacted starting material (i.e., the compound of formula (Ia)).

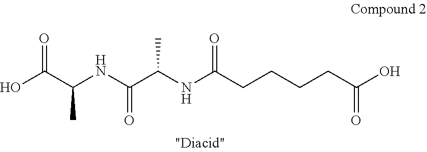 [SMILES: C[C@H](NC(=O)[C@H](C)NC(=O)CCCCC(=O)O)C(=O)O]

In a second embodiment, the present invention provides a method of preparing a compound of formula (Va):

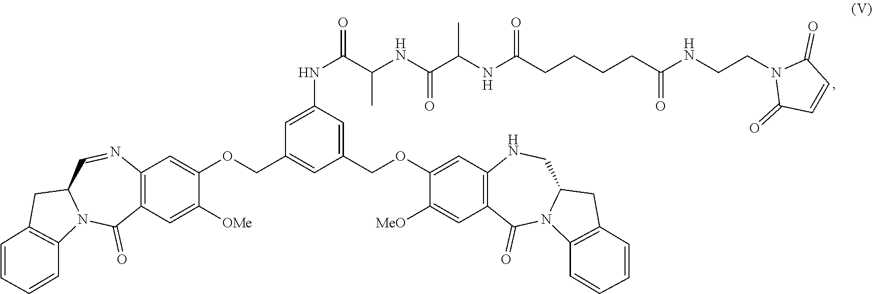 [SMILES: COc1cc2c(cc1OCc1cc(COc3cc4c(cc3OC)C(=O)N3c5ccccc5C[C@H]3CN4)cc(NC(=O)C(C)NC(=O)C(C)NC(=O)CCCCC(=O)NCCN3C(=O)C=CC3=O)c1)N=C[C@@H]1Cc3ccccc3N1C2=O]

comprising the step of reacting a compound formula (III)

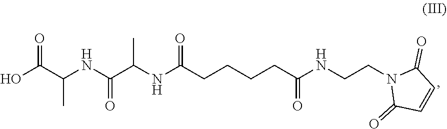 [SMILES: CC(NC(=O)C(C)NC(=O)CCCCC(=O)NCCN1C(=O)C=CC1=O)C(=O)O]

with a compound of formula (IVa):

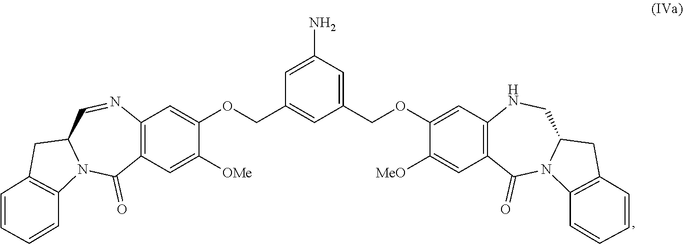 [SMILES: COc1cc2c(cc1OCc1cc(N)cc(COc3cc4c(cc3OC)C(=O)N3c5ccccc5C[C@H]3CN4)c1)N=C[C@@H]1Cc3ccccc3N1C2=O]

in the presence of an activating agent to form the compound of formula (Va).

In a 2^{nd} specific embodiment, the present invention provides a method of preparing a compound of formula (Va):

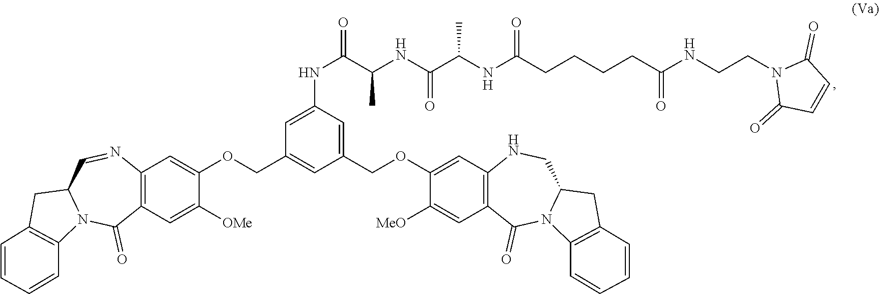 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O]

comprising the step of reacting a compound formula (IIIa)

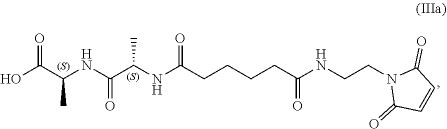

with a compound of formula (IVa):

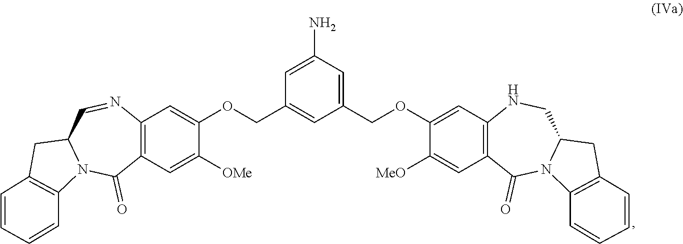 [SMILES: COc1cc2c(cc1OCc1cc(N)cc(COc3cc4c(cc3OC)C(=O)N3c5ccccc5C[C@H]3CN4)c1)N=C[C@@H]1Cc3ccccc3N1C2=O]

in the presence of an activating agent to form the compound of formula (Va).

The previously disclosed method for preparing the compound of formula (Va) utilizes N,N-diisopropylethylamine (DIPEA), HATU and HOAt. One drawback related to the old process is the inherent instability of the compound of formula (IIIa) under said reaction conditions, which may lead to the formation of by-product(s) that require additional purifications for their removal. Another key issue was the instability of the compound of formula (Va) during the aqueous work-up.

Other problem with the old process is the quality of the compound of formula (IIIa). As described above, the compound of formula (IIIa) prepared in the old process tends to be contaminated with the diacid impurity Compound 2, which can lead to additional impurities during the coupling reaction.

The present methods for preparing the compound of formula (Va) provide significant improvements in reaction rates and quality of the product, such as quick reaction times (e.g., complete within 1 hour), stable product (e.g., stable for up to 16 h without significant by-product formation), and high purity of the crude product (e.g., >90% purity of the compound of formula (Va) isolated from the reaction mixture before chromatography). For instance, the method of the present invention can eliminate the use of HOAt in the coupling reaction between the compound of formula (IIIa) and the compound of formula (IVa).

In one example of the present methods, it is observed that the presence of MeOH and lower reaction temperature (e.g., 10° C. or lower) can suppress oxidation of the compound of formula (IVa). In another example, the use of a quenching regent (e.g., methyl tert-butyl ether (MTBE)) in the present methods can directly precipitate the crude compound of formula (Va) out of the reaction solution, which can be readily filtered and isolated without the detrimental aqueous work-up associated with the previous method.

In a third embodiment, the present invention provides a method of preparing a compound of formula (V):

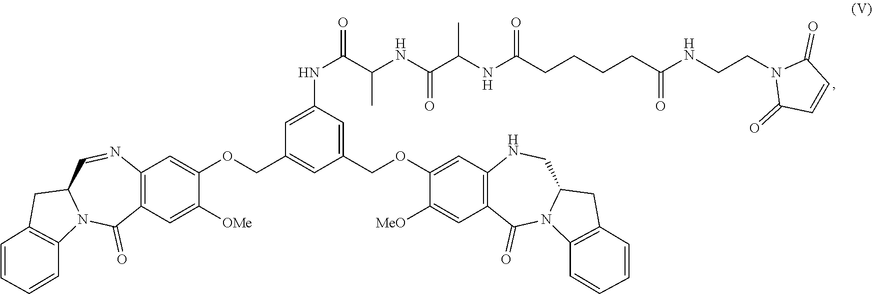 [SMILES: COc1cc2c(cc1OCc1cc(COc3cc4c(cc3OC)C(=O)N3c5ccccc5C[C@H]3CN4)cc(NC(=O)C(C)NC(=O)C(C)NC(=O)CCCCC(=O)NCCN3C(=O)C=CC3=O)c1)N=C[C@@H]1Cc3ccccc3N1C2=O]

comprising the steps of:
(i) reacting a compound of formula (I):

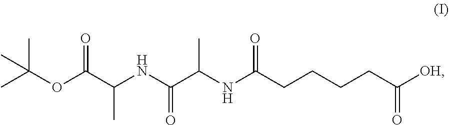 [SMILES: CC(NC(=O)CCCCC(=O)O)C(=O)NC(C)C(=O)OC(C)(C)C]

with a bisulfate salt (H₂SO₄ salt) of a compound of formula (b):

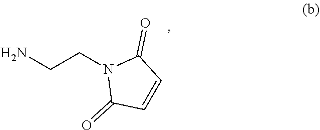

to form a compound of formula (II):

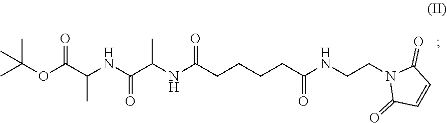 [SMILES: CC(NC(=O)CCCCC(=O)NCCN1C(=O)C=CC1=O)C(=O)NC(C)C(=O)OC(C)(C)C]

(ii) reacting the compound of formula (II) with a carboxylic acid deprotecting agent to form a compound of formula (III):

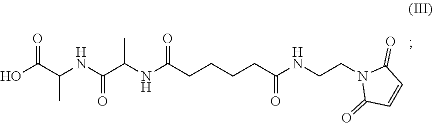 [SMILES: CC(NC(=O)C(C)NC(=O)CCCCC(=O)NCCN1C(=O)C=CC1=O)C(=O)O]

(iii) reacting the compound of formula (III) with a compound of formula (IVa):

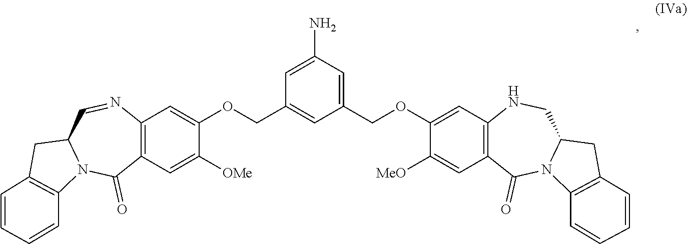 [SMILES: COc1cc2c(cc1OCc1cc(N)cc(COc3cc4c(cc3OC)C(=O)N3c5ccccc5C[C@H]3CN4)c1)N=C[C@@H]1Cc3ccccc3N1C2=O]

to form the compound of formula (V).

In a 3^{rd} specific embodiment, the present invention provides a method of preparing a compound of formula (Va):

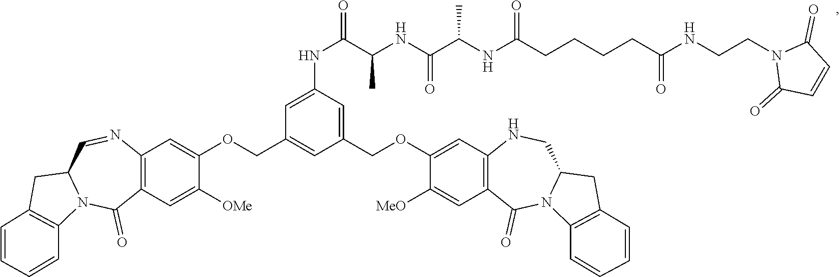 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O]

comprising the steps of:
(i) reacting a compound of formula (Ia):

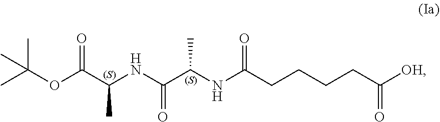

with a bisulfate salt (H₂SO₄ salt) of a compound of formula (b):

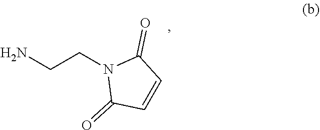

to form a compound of formula (IIa):

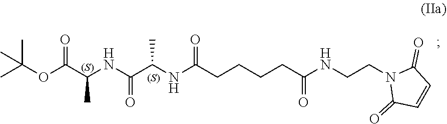

(ii) reacting the compound of formula (IIa) with a carboxylic acid deprotecting agent to form a compound of formula (IIIa):

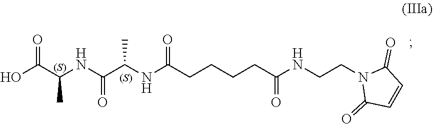

(iii) reacting the compound of formula (IIIa) with a compound of formula (IVa):

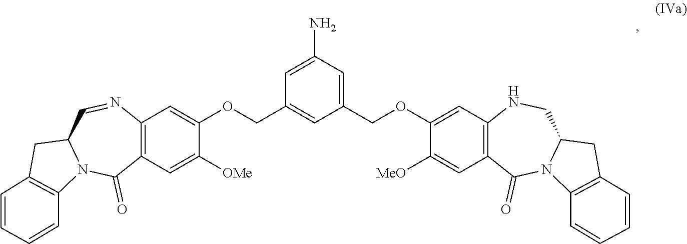 [SMILES: COc1cc2c(cc1OCc1cc(N)cc(COc3cc4c(cc3OC)C(=O)N3c5ccccc5C[C@H]3CN4)c1)N=C[C@@H]1Cc3ccccc3N1C2=O]

to form the compound of formula (Va).

In a fourth embodiment, for the method described in the first or third embodiment, or the 1^{st} or 3^{rd} specific embodiment, the reaction in step (i) between the compound of formula (I) or (Ia) or a salt thereof and the bisulfate salt (H₂SO₄) of the compound of formula (b) is carried out in the presence of an activating agent.

In a specific embodiment, the activating agent is a 2,4,6-trialkyl-1,3,5,2,4,6-trioxatriphosphorinane 2,4,6-trioxide, carbodiimide (e.g., N,N′-dicyclohexylcarbodiimide (DCC) or 1-ethyl-3-(3-dimethylaminopropyl)carbodiimide (EDC)), 1,1′-carbonyldiimidazole (CDI), a uronium, an activated ester, a phosphonium, 2-alkyl-1-alkylcarbonyl-1,2-dihydroquinoline, 2-alkoxy-1-alkoxycarbonyl-1,2-dihydroquinoline, or alkylchloroformate.

In another specific embodiment, the activating agent is 2,4,6-trialkyl-1,3,5,2,4,6-trioxatriphosphorinane 2,4,6-trioxide. In a more specific embodiment, the activating agent is 2,4,6-tripropyl-1,3,5,2,4,6-trioxatriphosphorinane 2,4,6-trioxide (T3P).

The activating agent can be used in any forms including powder, crystals, liquid or solution. In a specific embodiment, the activating agent is a solution in a solvent. Any suitable solvents can be used to make a solution of an activating agent. In a more specific embodiment, the activating agent is a solution of T3P in ethyl acetate (EtOAc). The concentration of an activating agent in a solvent is in the range of 0-100 wt %, 20-100 wt %, 30-100 wt %, 50-100 wt %, or 60-100 wt %. In a specific embodiment, the concentration of T3P in EtOAc is in the range of or 40-60 wt %.

Any suitable amount of the activating agent can be used in the reaction between the compound of fomula (I) or (Ia) or a salt thereof and the bisulfate salt (H₂SO₄) of the compound of formula (b). In one embodiment, between 1.0 and 5.0 molar equivalents of the activating agent (e.g., T3P) relative to the amount of the compound of formula (I) or (Ia) is used in the reaction. In a specific embodiment, 1.0-3.0, 2.0-3.0, 1.5-2.5, or 2.0-4.0 molar equivalent of T3P relative to the amount of the compound of formula (I) or (Ia) is used. In a more specific embodiment, 2.0 equivalent of T3P is used.

In one embodiment, the reaction between the compound of formula (I) or (Ia) or a salt thereof and the bisulfate salt (H₂SO₄) of the compound of formula (b) is carried out in the presence of a base. In one embodiment, the base is a non-nucleophilic base. Exemplary non-nucleophilic bases include, but are not limited to, triethylamine, imidazole, N,N-diisopropylethylamine, pyridine, 2,6-lutidine, dimethylformamide, 1,8-diazabicyclo[5.4.0]undec-7-ene (DBU), or tetramethylpiperidine. In a specific embodiment, the base is triethylamine or N,N-diisopropylethylamine. In another specific embodiment, the base is triethylamine.

In another embodiment, the reaction between the compound of formula (I) or (Ia) or a salt thereof and the bisulfate salt (H₂SO₄) of the compound of formula (b) is carried out in the presence of an activating agent described above and a base described above. In a specific embodiment, the reaction is carried out in the presence of 2,4,6-tripropyl-1,3,5,2,4,6-trioxatriphosphorinane 2,4,6-trioxide (T3P) as the activating agent and triethylamine or N,N-diisopropylethylamine as the base. In another specific embodiment, the reaction is carried out in the presence of 2,4,6-tripropyl-1,3,5,2,4,6-trioxatriphosphorinane 2,4,6-trioxide (T3P) and triethylamine.

The reaction between the compound of formula (I) or (Ia) or a salt thereof and the bisulfate salt (H₂SO₄) of the compound of formula (b) can be carried out in any suitable organic solvent(s). In one embodiment, the reaction is carried out in dichloromethane.

In another embodiment, the reaction between the compound of formula (I) or (Ia) or a salt thereof and the bisulfate salt (H₂SO₄) of the compound of formula (b) is carried out under an inert atmosphere. In a specific embodiment, the inert atmosphere is achieved by degassing the reaction solutions and purging the reaction vessel with nitrogen or argon.

The reaction between the compound of formula (I) or (Ia) or a salt thereof and the bisulfate salt (H₂SO₄) of the compound of formula (b) can be carried out at a suitable temperature. In some embodiments, the reaction is carried out at a temperature between 0° C. and 50° C., between 0° C. and 25° C., between 2° C. and 23° C., between 3° C. and 20° C., between 0° C. and 5° C. or between 0° C. and 3° C. In more specific embodiments, the reaction is carried out at a temperature between 0° C. and 20±3° C.

In a fifth embodiment, for the method described in the first or third embodiment, or the 1^{st} or 3^{rd} specific embodiment, the compound of formula (II) or (IIa) is purified by solvent extraction with an organic solvent to form an organic phase comprising the compound of formula (II) or (IIa) followed by one or more aqueous wash of the organic phase, wherein at least one of the aqueous wash is carried out with an aqueous bicarbonate solution. In a specific embodiment, the aqueous bicarbonate solution is an aqueous NaHCO₃ solution. The concentration of the aqueous bicarbonate solution is in the range of 0.0-10.0 mol/L (M). In some embodiments, the concentration is in the range of 0.0-5.0M, 0.5-4.0M, 1.0-3.0M, 1.0-2.0M, or 0.5-1.5M. In a more specific embodiment, the aqueous bicarbonate solution is 1M aqueous NaHCO₃ solution.

In a sixth embodiment, for the method described in the first or third embodiment or the 1^{st} or 3^{rd} specific embodiment, any suitable carboxylic acid deprotecting agent can be used in step (ii). In certain embodiments, an acid can be used to remove the tert-butyl ester protecting group. Exemplary acids include, but are not limited to, formic acid, acetic acid, trifluoroacetic acid, hydrochloric acid, and phosphoric acid. In a specific embodiment, trifluoroacetic acid is used as the carboxylic acid deprotecting agent.

In one embodiment, the deprotection reaction can be carried out in any suitable organic solvent(s). Exemplary organic solvents include, but are not limited to, DMF, CH₂Cl₂, dichloroethane, THF, dimethylacetamide, methanol, ethanol, etc. In a specific embodiment, the deprotection reaction is carried out in dichloromethane. The deprotection reaction can be carried out at a suitable temperature, for example, at a temperature between 0° C. and 50° C., between 0° C. and 25° C., between 0° C. and 10° C., between 15° C. and 25° C. or between 20° C. and 25° C.

In a seventh embodiment, for the method described in the second or third embodiment, or the 2^{nd} or 3^{rd} specific embodiment, the reaction between the compound of formula (III) or (IIIa) or a salt thereof and the compound of formula (IVa) or a salt thereof is carried out in the presence of an activating agent.

In a specific embodiment, the activating agent is a 2,4,6-trialkyl-1,3,5,2,4,6-trioxatriphosphorinane 2,4,6-trioxide, 1-[bis(dimethylamino)methylene]-1H-1,2,3-triazolo[4,5-b]pyridinium 3-oxid hexafluorophosphate (HATU), 1-hydroxy-7-azabenzotriazole or 1H-[1,2,3]triazolo[4,5-b]pyridin-1-ol (HOAt), carbodiimide (e.g., N,N′-dicyclohexylcarbodiimide (DCC) or 1-ethyl-3-(3-dimethylaminopropyl)carbodiimide (EDC)), 1,1′-carbonyldiimidazole (CDI), a uronium, an activated ester, a phosphonium, 2-alkyl-1-alkylcarbonyl-1,2-dihydroquinoline, 2-alkoxy-1-alkoxycarbonyl-1,2-dihydroquinoline, or alkylchloroformate, or a combination thereof. In a more specific embodiment, the activating agent is 1-[bis(dimethylamino)methylene]-1H-1,2,3-triazolo[4,5-b]pyridinium 3-oxid hexafluorophosphate (HATU).

Any suitable amount of the activating agent can be used in the reaction between the compound of formula (III) or (IIIa) or a salt thereof and the compound of formula (IVa) or a salt thereof. In one embodiment, between 1.0 and 5.0 molar equivalents of HATU relative to the amount of the compound of formula (IVa) is used in the reaction. In a specific embodiment, 1.0-2.0, 1.2-1.7, or 1.3-1.6 molar equivalents of HATU is used. In a specific embodiment, 1.2, 1.3, 1.4, 1.5, 1.6 or 1.7 molar equivalents of HATU is used. In a more specific embodiment, 1.5 equivalent of HATU is used.

In one embodiment, the reaction between the compound of formula (III) or (IIIa) or a salt thereof and the compound of formula (IVa) or a salt thereof is carried out in the presence of a base. In one embodiment, the base is a non-nucleophilic base. Exemplary non-nucleophilic bases include, but are not limited to, triethylamine, imidazole, N,N-diisopropylethylamine (or Hunig's base), pyridine, 2,6-lutidine, 1,8-diazabicyclo[5.4.0]undec-7-ene (DBU), or tetramethylpiperidine. In a specific embodiment, the base is triethylamine or N,N-diisopropylethylamine. In another specific embodiment, the base is N,N-diisopropylethylamine.

In another embodiment, the reaction between the compound of formula (III) or (IIIa) or a salt thereof and the compound of formula (IVa) or a salt thereof is carried out in the presence of an activating agent described above and a base described above. In a specific embodiment, the reaction is carried out in the presence of HATU as the activating agents and N,N-diisopropylethylamine as the base.

In a more specific embodiment, no HOAt is added to the reaction between the compound of formula (III) or (IIIa) or a salt thereof and the compound of formula (IVa) or a salt thereof.

In another embodiment, the reaction between the compound of formula (III) or (IIIa) or a salt thereof and the compound of formula (IVa) or a salt thereof is carried out under an inert atmosphere. In a specific embodiment, the inert atmosphere is achieved by degassing the reaction solvent and purging the reaction vessel with nitrogen or argon.

The reaction between the compound of formula (III) or (IIIa) or a salt thereof and the compound of formula (IVa) or a salt thereof can be carried out in any suitable organic solvent(s). In one embodiment, the reaction is carried out in dichloromethane. In another embodiment, the reaction between the compound of formula (III) or (IIIa) and the compound of formula (IVa) is carried out in the presence of an alcohol. In a specific embodiment, the alcohol is methanol.

In another embodiment, the reaction between the compound of formula (III) or (IIIa) or a salt thereof and the compound of formula (IVa) or a salt thereof is carried out in a mixture of dichloromethane and methanol. The volume ratio of dichloromethane to methanol used in the reaction is in the range of 1:10 to 10:1. In some embodiments, the volume ratio of dichloromethane to methanol is in the range of 1:5 to 8:1, 1:2 to 6:1, 1:1 to 5:1, 2:1 to 4:1, or 3:1 to 4:1. In a specific embodiment, the volume ratio of dichloromethane to methanol is 15:4.

The reaction between the compound of formula (III) or (IIIa) or a salt thereof and the compound of formula (IVa) or a salt thereof can be carried out at a suitable temperature. In some embodiments, the reaction is carried out at a temperature between 0° C. and 50° C., between 0° C. and 30° C., between 5° C. and 25° C., or between 10° C. and 15° C. In more specific embodiments, the reaction is carried out at 10° C.±5° C.

In an eighth embodiment, for the method described in the second or third embodiment or the 2^{nd} or 3^{rd} specific embodiment, the reaction between the compound of formula (III) or (IIIa) or a salt thereof and the compound of formula (IVa) or a salt thereof is quenched with methyl tert-butyl ether (MTBE). In one embodiment, the reaction between the compound of formula (III) or (IIIa) or a salt thereof and the compound of formula (IVa) or a salt thereof is cooled to a temperature between 0° C. and 5° C. and added the quenching reagent MTBE.

In another embodiment, the quenching reagent MTBE precipitates the crude product of the compound of formula (V) or (Va) out of the reaction mixture of the reaction between the compound of formula (III) or (IIIa) or a salt thereof and the compound of formula (IVa) or a salt thereof. In a specific embodiment, the crude product of the compound of formula (V) or (Va) is isolated from the reaction mixture by filtration. In particular, the isolated crude product comprises the compound of formula (V) or (Va) with a HPLC purity in the range of 90% to 100%.

In one embodiment, no aqueous workup of the reaction mixture is carried out after the reaction between the compound of formula (III) or (IIIa) or a salt thereof and the compound of formula (IVa) or a salt thereof.

In a ninth embodiment, the present invention provides a method of preparing a compound of formula (VIIa):

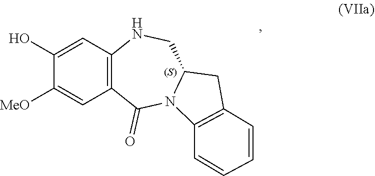

comprising the step of reacting a compound of formula (VIa):

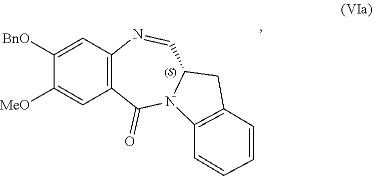

with H₂ in the presence of a catalyst.

Any suitable catalysts can be used for the reaction between the compound of formula (VIa) or a salt thereof and H₂. In one embodiment, the catalyst is a Raney nickel catalyst (e.g., Raney® Nickel (40-60 mesh), Type W6, Ni content ˜90%). In the previously disclosed process, it requires two-step reactions (debenzylation and reduction reactions) from benzyl monomer (i.e., the compound of formula (VIa)) to TBDI (i.e., the compound of formula (VIIa)). The method of present disclosure with the use of a catalyst (e.g., a nickel catalyst) completes two transformations (reductions) in one step, which is operationally simpler with improved throughput.

Suitable amount of the catalyst can be used. In one embodiment, 0.1-10.0, 1.0-10.0, 1.0-5.0, 3.0 to 5.0, 3.0-3.5, 5.0-10.0 or 6.0-7.0 molar equivalents of the catalyst relative to the compound of formula (VIa) can be used.

The reaction between the compound of formula (VIa) or a salt thereof and H₂ in the presence of a catalyst can be carried out in in any suitable organic solvent(s). Exemplary organic solvents include, but are not limited to, MeOH, EtOH, THF, propanol, isopropanol, n-butanol, t-butanol, dioxane, diethyl ether, tert-butyl methyl ether, etc. In some embodiment, the reaction is carried out in MeOH, EtOH or THF. In a specific embodiment, the reaction is carried out in MeOH.

The reaction between the compound of formula (VIa) or a salt thereof and H₂ in the presence of a catalyst can be carried out at a suitable temperature. In some embodiments, the reaction is carried out at a temperature between 0° C. and 100° C., between 20° C. and 80° C., between 30° C. and 70° C., between 40° C. and 60° C., or between 40° C. and 50° C. In more specific embodiments, the reaction is carried out at a temperature between 40° C. and 60° C. or between 45° C. and 55° C.

In some embodiment, the method in the ninth embodiment further comprises a step of contacting the reaction mixture with a metal scavenger (e.g., a silica-based scavenger) to remove Nickel. In a more specific embodiment, the silica-based scavenger is silicathiol (iMoLbox-LMat-NH01).

In another embodiment, the compound of formula (VIIa) is purified by crystallization. Any suitable solvents can used for the crystallization. In some embodiments, the crystallization is carried out in MeOH and water. In some embodiments, the volume ratio of MeOH to water is 1:10 to 10:1, 1:10 to 1:5, or 1:3 to 1:5. In some embodiments, the volume ratio of MeOH to water is 1:4. In some embodiments, the crystallization is carried by heating the compound of formula (VIIa) in the mixture of MeOH and water to an elevated temperature (e.g., at a temperature between 40° C. and 80° C., between 50° C. and 70° C., between 60° C. and 70° C., or between 65° C. and 70° C.) to fully dissolve the compound, followed by slow cooling (e.g., to room temperature or to a temperature between 0° C. and 10° C. or between 0° C. and 5° C.) to allow crystallization to occur.

The method of the present invention for preparing the compound of formula (VIIa) is a more efficient process as compared with the previously disclosed method (e.g., WO 2010/091150), in which the compound of formula (VIIa) was obtained in two-step reactions. In particular, in step 1, the benzyl group was removed using transfer hydrogenation employing cyclohexadiene as the hydrogen source to generate the compound of formula (d); and in step 2, the compound of formula (d) was treated with borane to reduce the imine functionality to afford the compound of formula (VIIa).

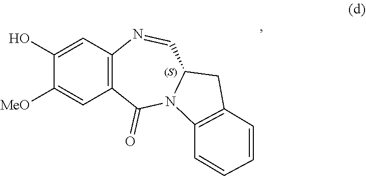

In a tenth embodiment, the present invention provides a method of preparing a compound of formula (VIIIa):

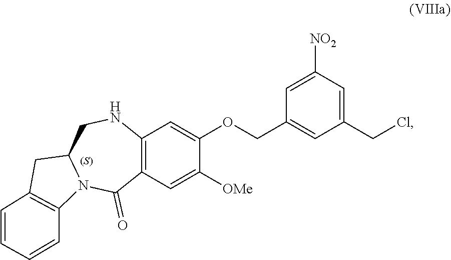

comprising the step of reacting a compound of formula (VIIa):

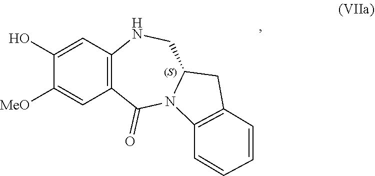

with a compound of formula (c):

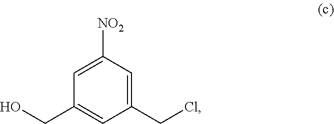 [SMILES: O=[N+]([O-])c1cc(CO)cc(CCl)c1]

to form the compound of formula (VIIIa).

In one embodiment, the reaction between the compound of formula (VIIa) or a salt thereof and the compound of formula (c) is carried out in the presence of an alcohol activating agent and an azodicarboxylate or an azodicarbonamide. In a specific embodiment, the alcohol activating agent is tributylphosphine, triphenylphosphine (PPh₃), or 2-bis(diphenylphosphino)ethane (DPPE). In another specific embodiment, the azodicarboxylate or azodicarbonamide is diethyl azodicarboxylate (DEAD), diisopropyl azodicarboxylate (DIAD), 1,1′-(azodicarbonyl)dipiperidine (ADDP), 1,1′-azodicarbonyldimorpholide (ADDM), 1,1′-azodicarbonyldiglyme (ADDG) and di-tert-butyl azodicarboxylate (DBAD). In a specific embodiment, the alcohol activating agent is 2-bis(diphenylphosphino)ethane (DPPE) and the azodicarboxylate or azodicarbonamide is 1,1′-(azodicarbonyl)dipiperidine (ADDP). In certain embodiments, 1.0-5.0, 1.0-2.0, 1.3-1.7 or 1.4-1.6 molar equivalents of the alcohol activating agent (e.g., DPPE) relative to the compound of formula (VIIa) and 1.0-5.0, 1.0-2.0, 1.3-1.7 or 1.4-1.6 molar equivalents of azodicarboxylate or azodicarbonamide (e.g., ADDP) relative to the compound of formula (VIIa) are used in the reaction.

The reaction between the compound of formula (VIIa) or a salt thereof and the compound of formula (c) can be carried out in in any suitable organic solvent(s). In a specific embodiment, the reaction is carried out in THF.

The reaction between the compound of formula (VIIa) or a salt thereof and the compound of formula (c) can be carried out at a suitable temperature. In some embodiments, the reaction is carried out at a temperature between 0° C. and 30° C., between 0° C. and 20° C., between 0° C. and 15° C., between 5° C. and 15° C., or between 5° C. and 20° C. In more specific embodiments, the reaction is carried out at a temperature between 5° C. and 15° C.

In some embodiments, the compound of formula (VIIIa) is purified by chromatography. In some embodiments, the compound of formula (VIIIa) is purified by crystallization. In some embodiments, the compound of formula (VIIIa) is crystallized as an acid addition salt. Any suitable acids can be used in the crystallization of the compound of formula (VIIIa). The suitable acids include, but are not limited to, (+)—O,O′-di-p-toluoyl-D-tartaric acid (D-(+)-DTTA), (−)—O, O′-di-p-toluoyl-L-tartaric acid (L-(−)-DTTA), sulfuric acid, phosphoric acid, hydrogen chloride, hippuric acid, benzenesulfonic acid, benzoic acid, D-tartaric acid, succinic acid, toluenesulfonic acid, camphorsulfonic acid, and methanesulfonic acid. In a specific embodiment, the acid is (+)—O, O′-Di-p-toluoyl-D-tartaric acid (D-(+)-DTTA). In another specific embodiment, the acid is sulfuric acid.

The present methods for preparing the compound of formula (VIIIa) is a more efficient process as compared to the previously disclosed process using triphenylphosphine (PPh₃) as the alcohol activating agent and DIAD as the azodicarboxylate or azodicarbonamide. The old process yields a lot of reaction by-products, which requires cumbersome purifications. For example, triphenylphosphine oxide by-product formed from PPh₃ is difficult to remove even after chromatography. The present invention has found that replacement of PPh₃ and DIAD with 1,2-bis(diphenylphosphino)ethane (DPPE) and 1,1′-(azodicarbonyl)dipiperidine (ADDP) can facilitate the removal of reaction by-products and provide product in high purity. For example, the mono oxide of DPPE by-product is highly insoluble in the reaction solvent and can be easily filtered off; and the reduced form of ADDP by-product is water soluble and can be readily removed by an aqueous wash.

In an eleventh embodiment, the method of the tenth embodiment further comprises the step of reacting the compound of formula (VIIIa) with a compound of formula (d):

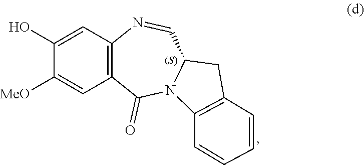

to form a compound of formula (IXa):

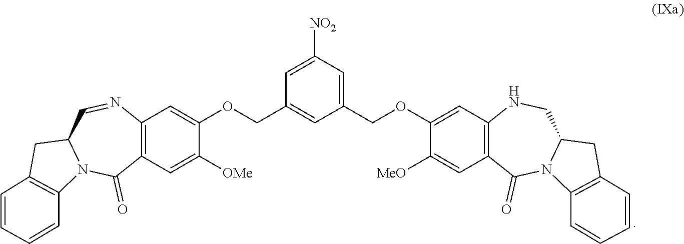 [SMILES: COc1cc2c(cc1OCc1cc(COc3cc4c(cc3OC)C(=O)N3c5ccccc5C[C@H]3CN4)cc([N+](=O)[O-])c1)N=C[C@@H]1Cc3ccccc3N1C2=O]

In one embodiment, the reaction between the compound of formula (VIIIa) and the monomer compound of formula (d) is carried out in the presence of a base. In one embodiment, the base is sodium carbonate, potassium carbonate, cesium carbonate, sodium hydride, or potassium hydride. In a specific embodiment, the base is potassium carbonate. In another embodiment, the reaction between the compound of formula (VIIIa) and the monomer compound of formula (d) further comprises potassium iodide. In a specific embodiment, the reaction between the compound of formula (VIIIa) and the monomer compound of formula (d) is carried out in the presence of potassium carbonate and potassium iodide.

The reaction between the compound of formula (VIIIa) or a salt thereof and the compound of formula (d) can be carried out in in any suitable organic solvent(s). Exemplary organic solvents include, but are not limited to, DMF, dimethylacetamide (DMA), MeCN, THF, dichloromethane, etc. In a specific embodiment, the reaction is carried out in DMF or DMA.

The reaction between the compound of formula (VIIIa) or a salt thereof and the compound of formula (d) can be carried out at a suitable temperature, for example, at a temperature between 0° C. and 50° C., between 0° C. and 30° C., between 0° C. and 25° C., between 0° C. and 10° C., between 15° C. and 25° C. or between 20° C. and 25° C. In a specific embodiment, the reaction is carried out at a temperature between 20° C. and 25° C.

In some embodiment, the compound of formula (IXa) is purified by precipitation. The precipitation can be carried out in any suitable solvents, e.g., a mixture of acetonitrile and water. In some embodiment, the ratio of the acetonitrile and water is 1:1 (v/v). The compound of formula (IXa) purified by the precipitation method has the purity in the range of 85-90% and can be used directly for the next step without cumbersome chromatography purification.

In a twelfth embodiment, the method of the eleventh embodiment further comprises the step of reacting the compound of formula (IXa) with a reducing agent to form a compound of formula (IVa):

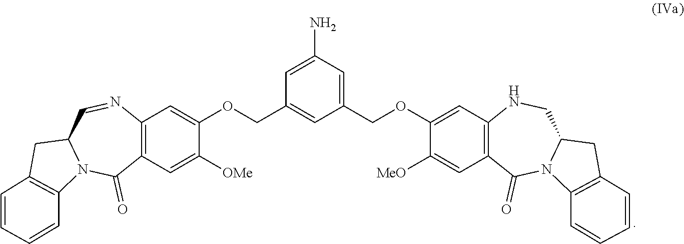 [SMILES: COc1cc2c(cc1OCc1cc(N)cc(COc3cc4c(cc3OC)C(=O)N3c5ccccc5C[C@H]3CN4)c1)N=C[C@@H]1Cc3ccccc3N1C2=O]

Any suitable reducing agent that can convert a nitro (—NO₂) group to an amine (—NH₂) group can be used for converting the compound of formula (IXa) to the compound of formula (IVa). In one embodiment, the reducing reagent is selected from the group consisting of: hydrogen gas, sodium hydrosulfite, sodium sulfide, stannous chloride, titanium (II) chloride, zinc, iron, and samarium iodide. In another embodiment, the reducing reagent is Fe/NH₄Cl, Zn/NH₄Cl, FeSO₄/NH₄OH, sulfur/NaBH₄ or Sponge Nickel. In a specific embodiment, the reducing agent is Fe/NH₄Cl.

In a thirteenth embodiment, for the method described in the tenth, eleventh, or twelfth embodiment, the compound of formula (c) is prepared by reacting a compound of formula (c1)

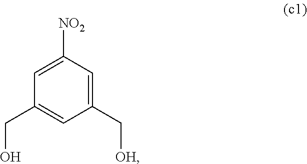 [SMILES: O=[N+]([O-])c1cc(CO)cc(CO)c1]

with hydrochloric acid in toluene.

All references cited herein and in the examples that follow are expressly incorporated by reference in their entireties.

### EXAMPLES

The following solvents, reagents, protecting groups, moieties and other designations may be referred to by their abbreviations in parenthesis:
eq=molar equivalent
V=volume
g=grams
h=hour
min=minutes
mg=milligrams
mL=milliliters
mmol=millimoles
T=temperature
LCMS=liquid chromatography mass spectrometry
HPLC=high Performance Liquid Chromatography
IPC=in-process control
MS=mass spectrometry
NEt₃=triethylamine
NMR=nuclear magnetic resonance spectroscopy
ACN or MeCN=acetonitrile
ADDP=1,1′-(azodicarbonyl)dipiperidine
DPPE=1,2-Bis(diphenylphosphino)ethane
DCM=dichloromethane
DIPEA=N,N-diisopropylethylamine or Hunig's base
DMAc=N,N-dimethylacetamide
EtOAc=ethyl acetate
IPAc=isopropyl acetate
HATU=1-[bis(dimethylamino)methylene]-1H-1,2,3-triazolo[4,5-b]pyridinium 3-oxid hexafluorophosphate
HOAt=1H-[1,2,3]triazolo[4,5-b]pyridin-1-ol or 1-hydroxy-7-azabenzotriazole
MTBE=Methyl tert-butyl ether
T3P=2,4,6-tripropyl-1,3,5,2,4,6-trioxatriphosphorinane 2,4,6-trioxide
TFA=trifluoroacetic acid
THF=tetrahydrofuran
v/v=volume/volume
wt=weight
wrt=with respect to
wt %=weight percent
w/w=weight/weight

### Example 1. Preparation of tert-butyl (6-((2-(2,5-dioxo-2,5-dihydro-1H-pyrrol-1-yl)ethyl)amino)-6-oxohexanoyl)-L-alanyl-L-alaninate (the compound of formula (IIa))

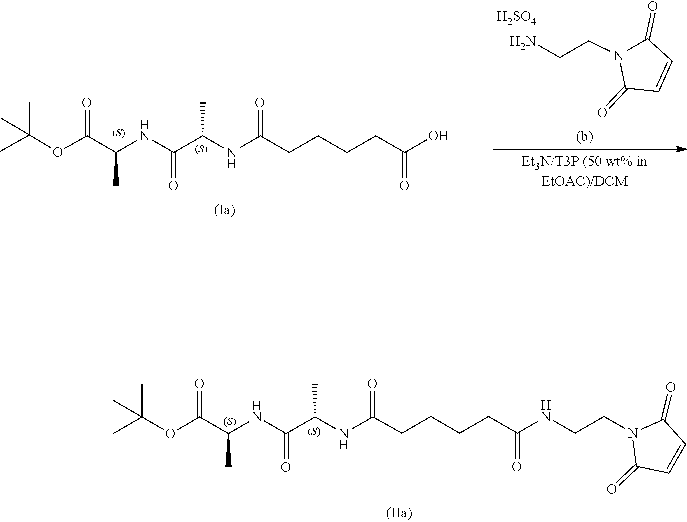

6-(((S)-1-(((S)-1-(tert-Butoxy)-1-oxopropan-2-yl)amino)-1-oxopropan-2-yl)amino)-6-oxohexanoic acid (the compound of formula (Ia)) (50.0 g, 145.2 mmol, 1.0 eq) was dissolved in dry DCM (800 mL) under a nitrogen atmosphere. 1-(2-Ethylamino)maleimide-bisulfate (the bisulfate salt of the compound of formula (b)) (36.3 g, 152.4 mmol, 1.05 eq) was added, along with DCM (100 mL) and the batch was cooled in an acetone/ice bath to T<5° C. A solution of NEt₃ (72 mL, 512.5 mmol, 3.53 eq) in DCM (225 mL) was added dropwise, maintaining T<5° C. The batch was cooled to T<2° C. and T3P (50 wt % in EtOAc, 173 mL, 290.4 mmol, 2.0 eq) was added dropwise over 20 min, maintaining T<3° C. The reaction mixture was stirred in the acetone/ice bath for 30 min, then warmed to ambient temperature and stirred overnight. The reaction was sampled at 3 h, 20 h, and 24 h. Reaction is complete if there is ≤2% starting material (the compound of formula (Ia)) with respect to product (the compound of formula (IIa)). At 24 h the reaction mixture was poured into stirring 75% sat. brine (750 mL) and the flask was rinsed with DCM (250 mL). The aqueous layer was removed, and the DCM layer was washed with 1 M HCl (500 mL). The organic layer was removed, and the aqueous layer was backextracted with DCM (200 mL). The combined organics were washed with 1 M NaHCO₃ (250 mL). The aqueous layer was removed, and the organic layer was washed with 75% saturated brine (275 mL). The organic was concentrated under reduced pressure to ˜20 vol with respect to theoretical product and polished filtered to remove NaCl. The filtrate (1.14 L) containing the compound of formula (IIa) was sampled for HPLC analysis (72% solution assay yield, 48.7 assay g, 104.4 mmol, HPLC purity=99.5 area %) and carried through to the next step as a solution in DCM.

### Example 2. Preparation of (6-((2-(2,5-dioxo-2,5-dihydro-1H-pyrrol-1-yl)ethyl)amino)-6-oxohexanoyl)-L-alanyl-L-alanine (the compound of formula (IIIa))

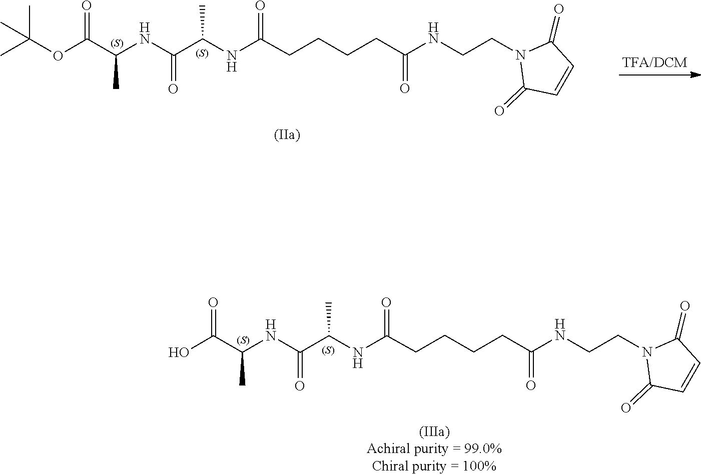

A solution of the compound of formula (IIa) (48.7 g, 104.4 mmol) in DCM (1.14 L) was placed in a cool water bath (T=18° C.) and TFA (244 mL, 3184 mmol, 30.5 eq) was added dropwise over 30 min, maintaining T=18-20° C. The reaction was stirred overnight at ambient temperature and sampled at 1.5 h and 20 h. Reaction is complete if there is ≤2% starting material (the compound of formula (IIa)) with respect to product (the compound of formula (IIIa)). At 20 h, the reaction was concentrated to a mobile oil, which was chased with DCM (3×100 mL). The resulting oil was dissolved in DCM (250 mL) and with stirring, MTBE (500 mL) was added slowly. The resulting suspension was concentrated under reduced pressure to ˜250 mL. MTBE (500 mL) was added and the suspension was concentrated to ˜250 mL. This was repeated two more times, at which point MTBE (500 mL) was added and the suspension was stirred at ambient temperature for 30 min. The solid was collected by filtration and washed with MTBE (50 mL). The solid was dried on the funnel overnight under a nitrogen atmosphere. The compound of formula (IIIa) was isolated as an off-white solid (41.1 g, 94% yield). Mass: m/z=411.2 [M+H]⁺; HPLC purity=99.0 area %.

### Example 3. Preparation of (3-(chloromethyl)-5-nitrophenyl)methanol (the Compound of Formula (c))

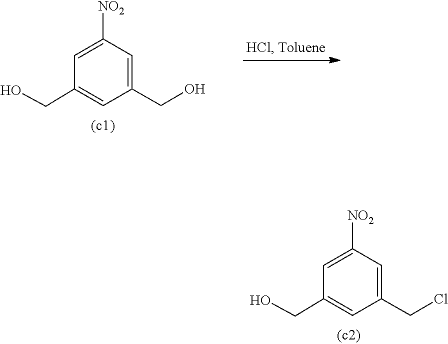 [SMILES: O=[N+]([O-])c1cc(CO)cc(CO)c1 ; O=[N+]([O-])c1cc(CO)cc(CCl)c1]

To a solution of 5-nitro-1,3-phenylene dimethanol (the compound of formula (c1)) (5.0 g, 1.0 eq.) in toluene (90.0 mL) was added concentrated hydrochloric acid (10.0 mL) dropwise over 10±2 minutes via addition funnel and the mixture was heated to 95±5° C. under nitrogen with stirring. The reaction was monitored by HPLC (IPC) for completion by analysis of 18 μL of the reaction mixture diluted into MeCN (1 mL). If there is ≤1% the compound of formula (c1) with respect to the compound of formula (c) the reaction is complete proceed to the next step. The HPLC showed the bulk of the reaction is complete within first 6 hours; elongated reaction times see an increase in the dichloride formation. After 16-24 hours, the reaction was cooled to 20±5° C. and washed with water (2×50.0 mL). The combined organic layers were collected and washed with saturated sodium bicarbonate (9 wt % of sodium bicarbonate in water; 50.0 mL) and concentrated to 50.0 mL on a rotary evaporator with bath temperature set at 40±5° C. The concentrate is cooled to 5±3° C. and aged for at least 2 hours to afford a white precipitate. Filter off the solid under vacuum and wash with toluene (2×25.0 mL). De-liquor the solid for a minimum of 1 hour under vacuum and transfer the solid to the oven and dry under vacuum at 25±5° C. until constant weight is achieved to afford the compound of formula (c) (3.67 g, 66.7% yield) as a white to yellow solid. HPLC purity=99.0 area %.

### Example 4. Preparation of (S)-9-hydroxy-8-methoxy-11,12,12a,13-tetrahydro-6H-benzo[5,6][1,4]diazepino[1,2-a]indol-6-one (the compound of formula (VIIa))

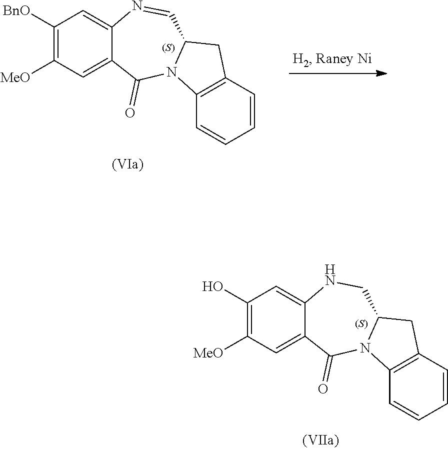

A solution of (S)-9-(benzyloxy)-8-methoxy-12a,13-dihydro-6H-benzo[5,6][1,4]diazepino[1,2-a]indol-6-one (the compound of formula (VIa)) (100 g, 0.260 moles) in MeOH (1988.6 mL) in a reactor was purged with argon and added Raney Ni (50 g, 0.849 moles, 3.26 equivalents). The reactor was swapped with H₂ under 50 psi three times, heated to 45-50° C. and continued to stir for 8-12 h. The reaction was monitored for completion by HPLC (IPC). If there was less than or equal to 1% the compound of formula (VIa) with respect to the compound of formula (VIIa), the reaction is considered complete. After cooling to 12˜25° C., the reaction mixture was filtered through 100 g diatomite (1-3×) to remove the spent catalyst. The filter wet cake was washed 3 times with 500 mL of MeOH. The filtrated was concentrated to 2 volumes (approximately 200 mL) below 50° C. under vacuum. To crystalize the product, the filtrate was heated to 65-70° C. and added 800 mL of water. The resultant mixture continued to stir for 30-60 min and then cooled at 0-5° C. for 1-2 h to precipitate the product out of solution. After filtration and washing the wet cake twice with 20% MeOH in water (100 g, 1 wt). The filter cake was dried under vacuum at 40-45° C. for 8-10 h to afford the desired product (the compound of formula (VIIa)) (57.1 g, 74% yield) as a light brown solid. Mass: m/z=297.3 [M+H]⁺; HPLC purity=98.6 area %.

### Example 5. Preparation of (S)-9-hydroxy-8-methoxy-11,12,12a,13-tetrahydro-6H-benzo[5,6][1,4]diazepino[1,2-a]indol-6-one (the compound of formula (VIIa)) with a silica-based scavenger

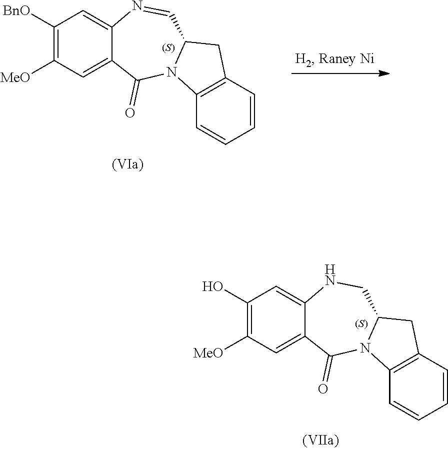

A solution of (S)-9-(benzyloxy)-8-methoxy-12a,13-dihydro-6H-benzo[5,6][1,4]diazepino[1,2-a]indol-6-one (the compound of formula (VIa)) (1 wt, 1 eq.) in MeOH (20 volumes) in a reactor was purged with argon and added Raney Nickel (Raney® Nickel (40-60 mesh), Type W6, Ni content ˜90%, 1 wt, 6.55 eq.). The reactor was swapped with H₂ under 50 psi three times, heated to 45-50° C. and continued to stir for 16-20 h. The reaction was monitored for completion by HPLC (IPC). If there was less than or equal to 1% the compound of formula (VIa) with respect to the compound of formula (VIIa), the reaction is considered complete. After cooling to 12˜25° C., the reaction mixture was filtered through a bed of Celite (1 wt). The filter wet cake was washed with MeOH (2×2 volumes). The washes were combined with the filtrate and added Silicathiol (iMoLbox-LMat-NH01, 0.5 wt) to remove Nickel. The suspension was heated to 45-55° C. and stirred for 5 hours. Then the suspension was filtered and washed with methanol (2×2 volumes). The organic layers were combined. To crystalize the product, the filtrate was heated to 65-70° C. and added water (8 volumes). The resultant mixture continued to stir for 30-60 min and then cooled at 0-5° C. for 1-2 h to precipitate the product out of solution. After filtration and washing the wet cake twice with a 1:4 mixture of MeOH:Water (2×1 volume). The filter cake was dried under vacuum at 40-45° C. for 40-50 h to afford the desired product (the compound of formula (VIIa)). Various scales up to 750 g of starting material were carried out using this preparation process. Observed yields of the compound of formula (VIIa) are 77%, 78%, and 74% with HPLC purity 98.6%, 98.2%, and 98.9%, respectively.

### Example 6. Preparation of (S)-9-((3-(chloromethyl)-5-nitrobenzyl)oxy)-8-methoxy-11,12,12a,13-tetrahydro-6H-benzo[5,6][1,4]diazepino[1,2-a]indol-6-one (the compound of formula (VIIIa))

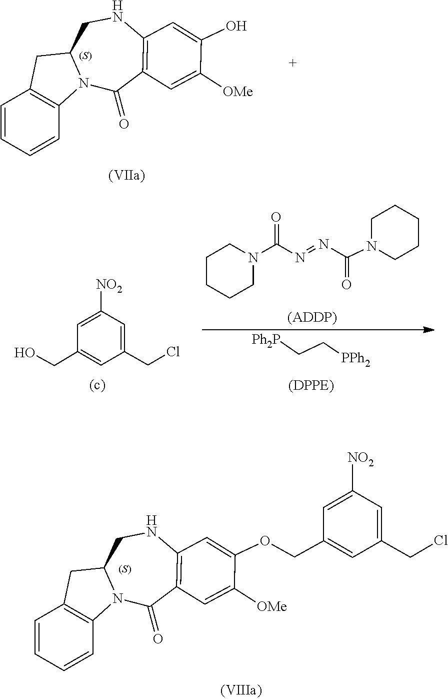

A solution of the compound of formula (VIIa) (8 g, 1.0 eq.), the compound of formula (c) (5.8 g, 1.08 eq.) and 1,2-Bis(diphenylphosphino)ethane (DPPE) (15.5 g, 1.46 eq.) in anhydrous THF (200 mL) was cooled to ≤10° C. under nitrogen and added a solution of 1,1′-(azodicarbonyl)dipiperidine (ADDP) (10.4 g, 1.54 eq.) in anhydrous THF (90 mL) via an addition funnel dropwise over approximately 4 minutes to afford an orange solution. The reaction mixture continued to stir at 10° C.±5° C. overnight (at least 16 hours) and monitored by HPLC (9-minute IPC method) for completion after at least 16 hours. The reaction is complete if there is ≤1.0% starting material (the compound of formula (VIIa)) with respect to product (the compound of formula (VIIIa)). At 16-18 hours, the reactions was diluted with IPAc (160 mL) and stirred for 1 hour at 10° C.±5° C. The suspension was filtered and washed with IPAc (80 mL) to remove the solid that was primarily the mono-oxide of DPPE, a by-product of the reaction. The filtrates were combined and concentrated to approximately (18 mL) under reduced pressure on a rotary evaporator, and then added IPAc (80 mL). The suspension was washed with water (80 mL) followed by 25% w/w brine (80 mL). Separate off the organic layer and hold the aqueous layer until the yield is known. The organic layer was concentrated to dryness under reduced pressure on a rotary evaporator to give the crude product, which was purified via silica gel column chromatography eluting with a gradient of 0-20% DCM/EtOAc over 40 minutes with a column load of 1 to 23 (23 g of silica per gram of crude product) to afford the compound of formula (VIIIa) (7.78 g, 60.7% yield) as a solid. HPLC purity=99.6 area %.

### Example 7. Preparation of (S)-8-methoxy-9-((3-((((S)-8-methoxy-6-oxo-11,12,12a,13-tetrahydro-6H-benzo[5,6][1,4]diazepino[1,2-a]indol-9-yl)oxy)methyl)-5-nitrobenzyl)oxy)-12a,13-dihydro-6H-benzo[5,6][1,4]diazepino[1,2-a]indol-6-one (the compound of formula (IXa))

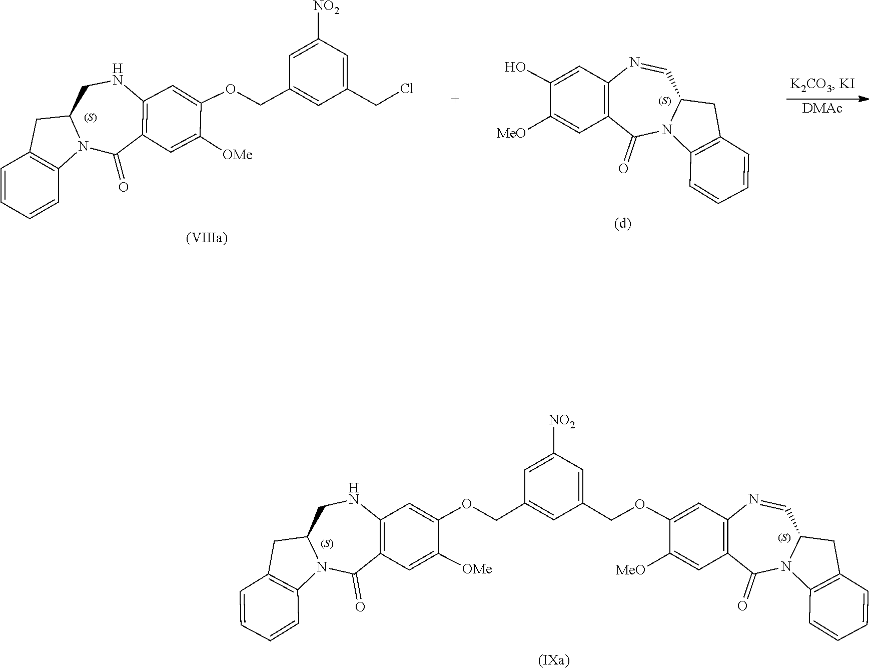

The compound of formula (VIIIa) (7.78 g, 1 eq.) was dissolved in DMAc (28 mL) and sparged with nitrogen for 15 minutes to remove dissolved oxygen which can lead to oxidation of the indolinobenzodiazepine ring system leading to formation of unwanted indole impurities. In another reactor, a solution of (S)-9-hydroxy-8-methoxy-12a,13-dihydro-6H-benzo[5,6][1,4]diazepino[1,2-a]indol-6-one (the compound of formula (d)) (6 g, 1.5 eq.), potassium iodide (1.35 g, 0.5 eq.), and potassium carbonate (4.48 g, 2.00 eq.) in DMAc (52 mL) was sparged with nitrogen at room temperature for at least 15 minutes prior to use to remove dissolved oxygen. The reactor was covered with aluminum foil to occlude incident light and the hood lights were also turned off during the reaction. The reaction mixture was heated to 35±5° C. under nitrogen and sparged with nitrogen for at least 30 minutes at this temperature. It is important to remove oxygen from the reaction to minimize unwanted by-products. The prepared degassed solution of the compound of formula (VIIIa) (7.78 g, 1 eq.) in DMAc (28 mL) was added into the reaction mixture via a syringe pump over approximately 1 hour. The reaction is monitored for completion by HPLC (IPC) after 3 hours. If there is ≤2.0% the compound of formula (VIIIa) with respect to the compound of formula (IXa) the reaction is complete. After completion, the reaction is cooled to 20° C.±5° C. and quenched by slowly adding a 50:50 mixture of MeCN:H₂O (320 mL) into the reaction mixture over at least 10 minutes. After stirring for 1 h at 20° C.±5° C., the solid was filtered off under vacuum on a polypropylene medium frit filter and washed sequentially with water (40 mL), 10% IPA:H₂O (40 mL) and finally 10% IPA/n-Heptane (40 mL). The solid on the filter was aspirated under vacuum for at least 1 hour to partially dry, then transfer to a vacuum oven and dry at ≤25° C. under full vacuum until constant weight is achieved to afford the compound of formula (IXa) (11.96 g, 100% yield) as a light orange to yellow solid. Solid often contains residual solvent even after reaching constant weight so the reaction is assumed to be quantitative for the purposes of calculating the stoichiometry in the next step. If corrected for assay the yield for the reaction is >80%. Mass: m/z=738.2 [M+H]⁺; HPLC purity=88.1 area %.

### Example 8. Preparation of (S)-9-((3-amino-5-((((S)-8-methoxy-6-oxo-11,12,12a,13-tetrahydro-6H-benzo[5,6][1,4]diazepino[1,2-a]indol-9-yl)oxy)methyl)benzyl)oxy)-8-methoxy-12a,13-dihydro-6H-benzo[5,6][1,4]diazepino[1,2-a]indol-6-one (the compound of formula (IVa))

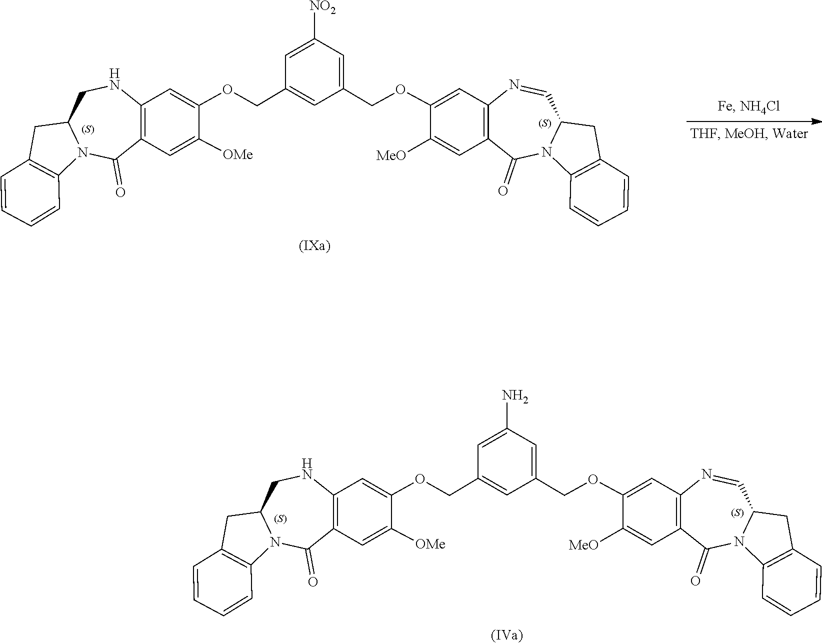

To a solution of the compound of formula (IXa) (20.00 g, 1 eq.) in tetrahydrofuran (250 mL), MeOH (34 mL), and deionized water (17 mL) was added ammonium chloride (15.23 g, 10.5 eq.). The reaction mixture was purged with nitrogen for approximately 5-15 minutes while heating to 40-45° C. Iron powder (extra pure reduced particle size about 10 μm, 8.48 g, 5.6 eq.) was added in one portion. The resultant mixture was heated to 60° C.±5° C. under nitrogen and monitored for completion by HPLC (IPC) after 4-6 hours. If there is ≤1% of the compound of formula (IXa) with respect to the compound of formula (IVa) the reaction is complete. After completion, the reaction was cooled to 20° C.±5° C. and added DCM (20 mL). The suspension was filtered through a pad of Celite® and the wet cake was washed with DCM (200 mL). The filtrate was concentrated to dryness under reduced pressure and re-dissolved in DCM (300 mL), which was followed by washing with deionized water (2×100 mL). The organic layer was collected and concentrated to dryness under reduced pressure. DCM (100 mL) was added to dissolve the dried residue and the resultant solution was concentrated to dryness under reduce pressure until no significant solvent condensing off the cooling finger/condenser to give the crude product as a brown solid. The crude product was purified via silica gel column chromatography eluting with MeOH/DCM (1/100, v/v) to MeOH/DCM (2/100, v/v) to afford the compound of formula (IVa) (11.88 g, 62% yield) as a light-yellow solid. HPLC purity=95.6 area %.

### Example 9. Preparation of N1-(2-(2,5-dioxo-2,5-dihydro-1H-pyrrol-1-yl)ethyl)-N6-((S)-1-(((S)-1-((3-((((S)-8-methoxy-6-oxo-11,12,12a,13-tetrahydro-6H-benzo[5,6][1,4]diazepino[1,2-a]indol-9-yl)oxy)methyl)-5-((((S)-8-methoxy-6-oxo-12a,13-dihydro-6H-benzo[5,6][1,4]diazepino[1,2-a]indol-9-yl)oxy)methyl)phenyl)amino)-1-oxopropan-2-yl)amino)-1-oxopropan-2-yl)adipamide (the compound of formula (Va))

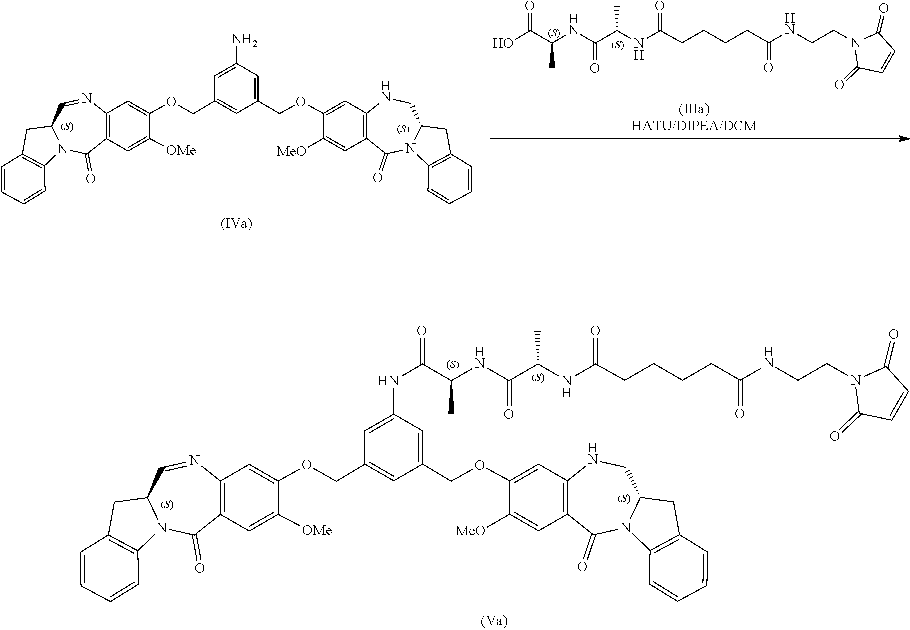

In a reactor, a solution of the compound of formula (IIIa) (3.95 g, 1.5 eq.) and HATU (3.66 g, 1.5 eq.) in anhydrous DCM (75 mL) was cooled to 10° C.±1° C. under nitrogen and added MeOH (20 mL). The compound of formula (IVa) (4.41 g, 1 eq.) was dissolved in anhydrous DCM (65 mL) under nitrogen and added DIPEA (1.64 mL, 1.5 eq.) and MeOH (20 mL) to afford a yellow solution, which was added to the reactor containing the previously-prepared mixture of the compound of formula (IIIa) and HATU in DCM and MeOH under nitrogen at 10° C.±5° C. A clear brown solution was observed. The reaction mixture continued to stir at 10° C.±2° C. under nitrogen and was monitor for completion by HPLC (IPC) (35-minute IPC method). When there is ≤1% starting material (the compound of formula (IVa)) relative to product (the compound of formula (Va)), the reaction is complete and proceed to the next step. At about 1 hour, the reaction was observed to be complete and cooled to 0° C.±2° C. under nitrogen. Pre-cooled (−10° C.) MTBE (350 mL) was added to precipitate the crude product out of the solution. The suspension was stirred at 0° C.±5° C. for 10 minutes with rigorous agitation. It was demonstrated that the suspension is stable for at least 20 hours at −20° C. The solid was filtered off under vacuum while maintaining a nitrogen atmosphere over the solid and the wet cake was washed with MTBE (150 mL) and n-Heptane (150 mL). The filtered solid was dried under vacuum for at least 2 hours with a positive nitrogen atmosphere above the cake to give the crude product (the compound of formula (Va)) (assay yield typically >80% and HPLC purity typically >92%), which was shown to be stable at room temperature on the filter for at least 16 hours. The crude product was dissolved in 1:1 mixture of DMA:MeCN (200 mL) and loaded into a pre-equilibrated RediSep Rf Gold C₁₈ column (Teledyne ISCO 69-2203-341) for purification. The column was equilibrated with 5% MeCN in water at a flow rate 150 mL/min and then eluted with 5% MeCN in water at a flow rate 14 mL/min for 5 column volumes, 50% MeCN in water at a flow rate 110 mL/min for 5 column volumes, and 100% MeCN at a flow rate 110 mL/min for 5 column volumes (detection/collection wavelength 254 nm). The combined fractions where the area % of the compound of formula (Va) is ≥90% were extracted with DCM (0.5 volumes relative to the total volume of the combined MeCN/water fractions) The DCM layer was separated then evaporated to dryness under vacuum. The residue was re-dissolved in anhydrous DCM (30 mL) at 20° C.±5° C. under nitrogen and while stirring, added MTBE (60 mL) to precipitate the solid, which was filtered under vacuum while maintaining a nitrogen atmosphere over the solid. The wet cake was washed with 1:2/DCM:MTBE (15 mL). De-liquor the solid under vacuum for at least 2 hours with a positive nitrogen atmosphere above the cake and transfer the solid to the vacuum oven and dry at ≤35° C. under full vacuum for at least 24 hours until constant weight is achieved to afford the desired product (the compound of formula (Va)) (4.45 g, 65% yield) as a yellow solid. HPLC purity=97.3 area %; Mass: m/z=1100.25 [M+H]⁺.

### Example 10. Purification of the Compound of Formula (VIIIa) Through Salt Formation

The crude product stream of Example 5 (˜66% pure by HPLC) was mixed with the stock solution shown in Table 1 below at room temperature. The resultant mixture was allowed to stir at ambient overnight. Where crystal formation was observed, they were isolated by filtration and analyzed by ¹H NMR and HPLC to assess the quality of the salt. Of the salts (Table 1) screened, the ditoluyl tartaric salt of the compound of formula (VIIIa) by far gave the best purity upgrade with the compound of formula (VIIIa) isolated in 98.7% purity. The sulfate was second best, giving the compound of formula (VIIIa) in 85.5% purity. With both the phosphate and hydrochloride salts affording the compound of formula (VIIIa) in ˜80% purity.

TABLE 1

| | | | | Ratio |
|---|---|---|---|---|
| | Volume | | | (Acid:the compound |
| Entry | (mL) | Acid | MW | of formula (VIIIa)) |
| 1 | 5 | Hippuric | 179.18 | 1.06 |
| 2 | 5 | Benzenesulfonic | 158.17 | 1.17 |
| 3 | 5 | Benzoic | 122.12 | 1.09 |
| 4 | 5 | D-tartaric | 150.09 | 1.11 |
| 5 | 5 | D-tartaric | 150.09 | 2.42 |
| 6 | 5 | Succinic | 118.09 | 1.22 |
| 7 | 5 | Succinic | 118.09 | 2.25 |
| 8 | 5 | toluenesulfonic•H₂O | 190.21 | 1.12 |
| 9 | 5 | phosphoric (>85%) | 98.00 | 1.53 |
| 10 | 20 | camphorsulfonic | 232.29 | 1.00 |
| 11 | 20 | methanesulfonic | 96.10 | 1.00 |
| 12 | 20 | sulfuric (conc.) | 98.07 | 1.00 |
| 13 | 20 | hydrogen chloride | | 1.00 |
| | | (5M in IPA) | | |
| 14 | 20 | Di-p-toluoyl-L-tartaric | 386.35 | 1.00 |
| | | acid | | |

### Example 11. Representative Salt Formation and Liberation of the Freebase

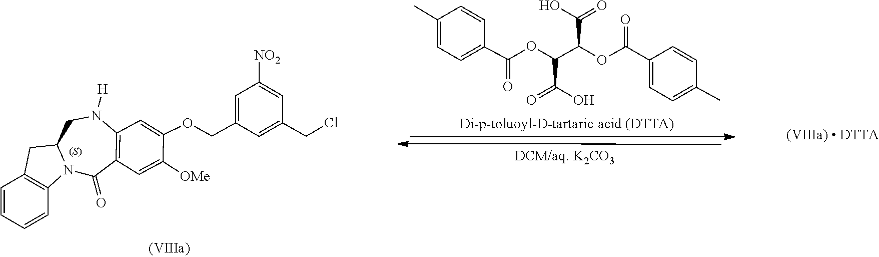

A solution of the compound of formula (VIIIa) (calculated 8.69 g/18.1 mmol by solution assay) in IPAc was azeotropically dried at ˜45° C./˜150 mmHg pressure) to a volume of ˜125 mL, the resulting solution was stirred at ambient while (+)—O,O′-di-p-toluoyl-D-tartaric acid (7.23 g, 18.7 mmol) was added as a solid. A solution initially formed but a suspension began to form within 5-10 min. The mixture was stirred overnight and then cooled to 0° C. The salt was isolated by filtration and washed with a cold mixture of 1:1 MTBE:IPAc (50 mL). A second wash with MTBE (50 mL at ambient) was applied and then the salt was dried on the filter under a nitrogen stream to afford the di-p-toluoyl-D-tartaric acid salt of the compound of formula (VIIIa) ((VIIIa)·DTTA) as fine, chalky light-yellow powder (9.45 g, 98.7 area % purity) in 60.3% yield.

The compound of formula (VIIIa) can be liberated from its salt by dissolving it in DCM and washing with an aqueous solution of potassium carbonate. A 500 mL flask with magnetic stir bar was charged with (VIIIa)·DTTA (7.59 g, 8.8 mmol) and DCM (80 mL). Agitation gave a yellowish suspension which was treated with 2.65 M aq. potassium carbonate solution. All solids dissolved within 1 min and the aqueous layer was diluted with water (40 mL). The lower organic layer was concentrated to afford high purity of the compound of formula (VIIIa) (4.21 g, 99.1 area % purity) in >99% yield.

What is claimed is:

1. A method of preparing a compound of formula (III): 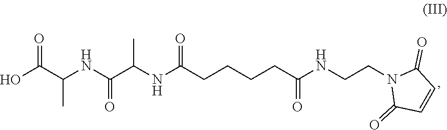 [SMILES: CC(NC(=O)C(C)NC(=O)CCCCC(=O)NCCN1C(=O)C=CC1=O)C(=O)O]
comprising the steps of
(i) reacting a compound of formula (I): 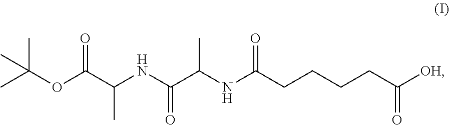 [SMILES: CC(NC(=O)CCCCC(=O)O)C(=O)NC(C)C(=O)OC(C)(C)C]
with a bisulfate salt (H₂SO₄ salt) of a compound of formula (b): 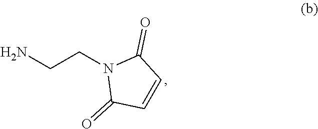
to form a compound of formula (II): 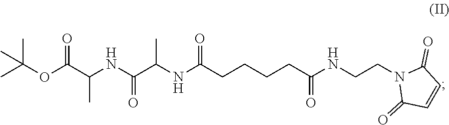 [SMILES: CC(NC(=O)CCCCC(=O)NCCN1C(=O)C=CC1=O)C(=O)NC(C)C(=O)OC(C)(C)C]
(ii) reacting the compound of formula (II) with a carboxylic acid deprotecting agent to form the compound of formula (III).

2. The method of claim 1, wherein the compound of formula (III) is represented by a compound of formula (IIIa) 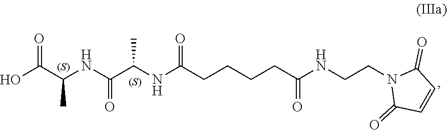
and the method comprises the steps of:
(i) reacting a compound of formula (Ia): 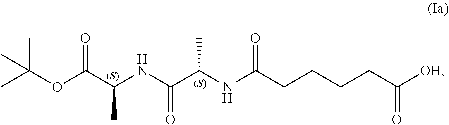
with the bisulfate salt (H₂SO₄ salt) of the compound of formula (b) to form a compound of formula (IIa): 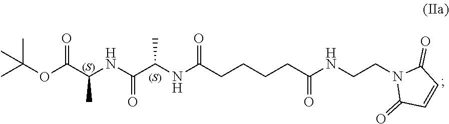
(ii) reacting the compound of formula (IIa) with a carboxylic acid deprotecting agent to form the compound of formula (IIIa).

3. The method of claim 1 or 2, wherein in step (i), the reaction between the compound of formula (I) or (Ia) and the bisulfate salt (H₂SO₄) of the compound of formula (b) is carried out in the presence of an activating agent.

4. The method of claim 3, wherein the activating agent is a 2,4,6-trialkyl-1,3,5,2,4,6-trioxatriphosphorinane 2,4,6-trioxide, carbodiimide, a uronium, an activated ester, a phosphonium, 2-alkyl-1-alkylcarbonyl-1,2-dihydroquinoline, 2-alkoxy-1-alkoxycarbonyl-1,2-dihydroquinoline, or alkylchloroformate.

5. The method of claim 4, wherein the activating agent is 2,4,6-tripropyl-1,3,5,2,4,6-trioxatriphosphorinane 2,4,6-trioxide (T3P).

6. The method of any one of claims 1-5, wherein in step (i), the reaction between the compound of formula (I) or (Ia) and the bisulfate salt (H₂SO₄) of the compound of formula (b) is carried out in the presence of a base.

7. The method of claim 6, wherein the base is triethylamine or N,N-diisopropylethylamine.

8. The method of any one of claims 3-7, wherein the reaction is carried out in dichloromethane.

9. The method of any one of claims 1-8, wherein the compound of formula (II) or (IIa) is purified by solvent extraction with an organic solvent to form an organic phase comprising the compound of formula (II) or (IIa) followed by one or more aqueous washes of the organic phase, wherein at least one of the aqueous washes is carried out with an aqueous bicarbonate solution.

10. The method of claim 9, wherein the aqueous bicarbonate solution is an aqueous NaHCO₃ solution.

11. The method of any one of claims 1-10, wherein the carboxylic acid deprotecting agent in step (ii) is trifluoroacetic acid (TFA).

12. A method of preparing a compound of formula (Va): 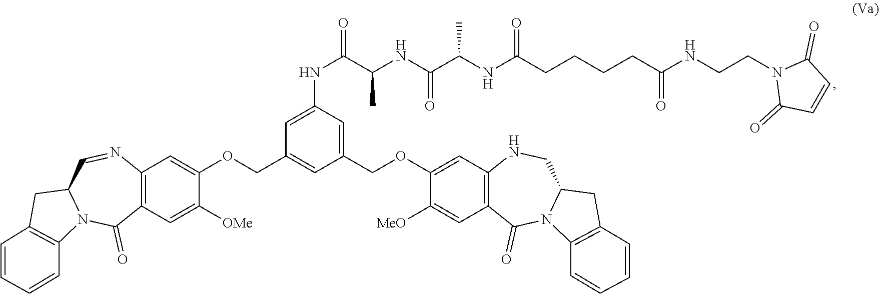 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O]
comprising the step of reacting a compound formula (IIIa) 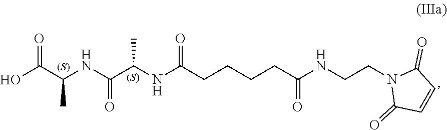
with a compound of formula (IVa): 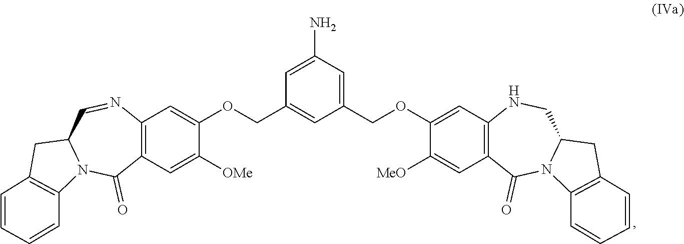 [SMILES: COc1cc2c(cc1OCc1cc(N)cc(COc3cc4c(cc3OC)C(=O)N3c5ccccc5C[C@H]3CN4)c1)N=C[C@@H]1Cc3ccccc3N1C2=O]
in the presence of an activating agent, in methanol to form the compound of formula (Va), wherein the activating agent is HATU.

13. The method of claim 12, wherein the reaction between the compound of formula (IIIa) and the compound of formula (IVa) is carried out in the presence of a base.

14. The method of claim 13, wherein the base is triethylamine or N,N-diisopropylethylamine.

15. The method of any one of claims 12-14, wherein the reaction is quenched with methyl tert-butyl ether (MTBE).

16. The method of any one of claims 12-15, wherein no HOAt is added to the reaction.

17. The method of any one of claims 12-16, wherein no aqueous workup of the reaction mixture is carried out.

18. A method of preparing a compound of formula (Va): 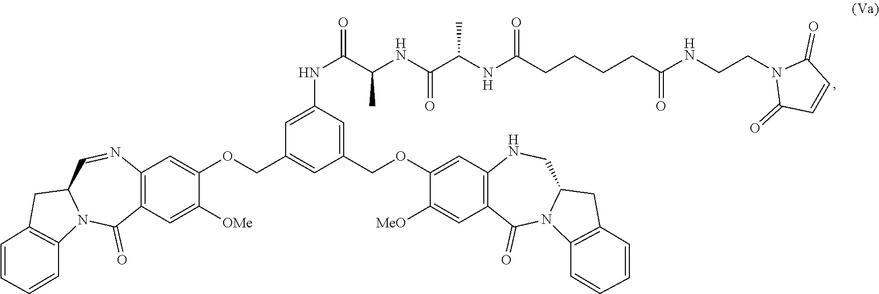 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O]
comprising the steps of:
(i) reacting a compound of formula (Ia): 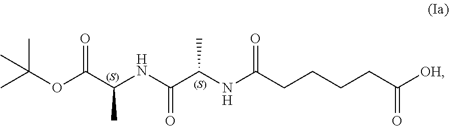
with a bisulfate salt (H₂SO₄ salt) of a compound of formula (b): 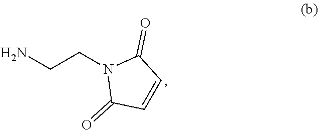
to form a compound of formula (IIa): 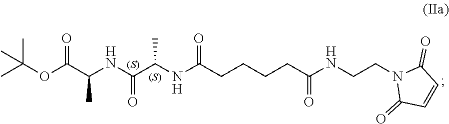
(ii) reacting the compound of formula (IIa) with a carboxylic acid deprotecting agent to form a compound of formula (IIIa): 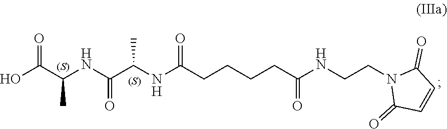
(iii) reacting the compound of formula (IIIa) with a compound of formula (IVa): 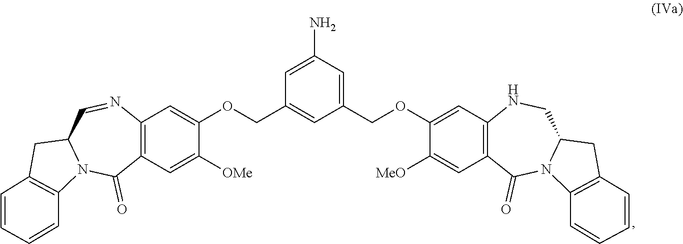 [SMILES: COc1cc2c(cc1OCc1cc(N)cc(COc3cc4c(cc3OC)C(=O)N3c5ccccc5C[C@H]3CN4)c1)N=C[C@@H]1Cc3ccccc3N1C2=O]
to form the compound of formula (Va).

19. The method of claim 18, wherein in step (i), the reaction between the compound of formula (Ia) and the bisulfate salt (H₂SO₄) of the compound of formula (b) is performed in the presence of an activating agent.

20. The method of claim 19, wherein the activating agent is a 2,4,6-trialkyl-1,3,5,2,4,6-trioxatriphosphorinane 2,4,6-trioxide, carbodiimide, a uronium, an activated ester, a phosphonium, 2-alkyl-1-alkylcarbonyl-1,2-dihydroquinoline, 2-alkoxy-1-alkoxycarbonyl-1,2-dihydroquinoline, or alkylchloroformate.

21. The method of claim 20, wherein the activating agent is 2,4,6-tripropyl-1,3,5,2,4,6-trioxatriphosphorinane 2,4,6-trioxide (T3P).

22. The method of any one of claims 18-21, wherein in step (i), the reaction between the compound of formula (Ia) and the bisulfate salt (H₂SO₄) of the compound of formula (b) is performed in the presence of a base.

23. The method of claim 22, wherein the base is triethylamine or N,N-diisopropylethylamine.

24. The method of any one of claims 18-23, wherein the compound of formula (IIa) is purified by solvent extraction with an organic solvent to form an organic phase comprising the compound of formula (IIa) followed by one or more aqueous wash of the organic phase, wherein at least one of the aqueous washes is carried out with an aqueous bicarbonate solution.

25. The method of claim 24, wherein the aqueous bicarbonate solution is an aqueous NaHCO₃ solution.

26. The method of any one of claims 18-25, wherein the carboxylic acid deprotecting agent in step (ii) is trifluoroacetic acid (TFA).

27. The method of any one of claims 18-26, wherein in step (iii), the reaction between the compound of formula (IIIa) and the compound of formula (IVa) is performed in the presence of an activating agent.

28. The method of claim 27, wherein the activating agent is a 2,4,6-trialkyl-1,3,5,2,4,6-trioxatriphosphorinane 2,4,6-trioxide, 1-[bis(dimethylamino)methylene]-1H-1,2,3-triazolo[4,5-b]pyridinium 3-oxid hexafluorophosphate (HATU), 1-hydroxy-7-azabenzotriazole or 1H-[1,2,3]triazolo[4,5-b]pyridin-1-ol (HOAt), carbodiimide (e.g., N,N′-dicyclohexylcarbodiimide (DCC) or 1-ethyl-3-(3-dimethylaminopropyl)carbodiimide (EDC)), 1,1′-carbonyldiimidazole (CDI), a uronium, an activated ester, a phosphonium, 2-alkyl-1-alkylcarbonyl-1,2-dihydroquinoline, 2-alkoxy-1-alkoxycarbonyl-1,2-dihydroquinoline, or alkylchloroformate, or a combination thereof.

29. The method of claim 28, wherein the activating agent is HATU.

30. The method of any one of claims 18-29, wherein in step (iii), the reaction between the compound of formula (IIIa) and the compound of formula (IVa) is carried out in the presence of a base.

31. The method of claim 30, wherein the base is triethylamine or N,N-diisopropylethylamine.

32. The method of any one of claims 18-31, wherein in step (iii), the reaction between the compound of formula (IIIa) and the compound of formula (IVa) is carried out in the presence of an alcohol.

33. The method of claim 32, wherein the alcohol is methanol.

34. The method of any one of claims 18-33, wherein the reaction in step (iii) is quenched with methyl tert-butyl ether (MTBE).

35. The method of any one of claims 18-34, wherein no HOAt is added to the reaction in step (iii).

36. The method of any one of claims 18-35, wherein no aqueous workup of the reaction mixture in step (iii) is carried out.

37. A method of preparing a compound of formula (VIIa): 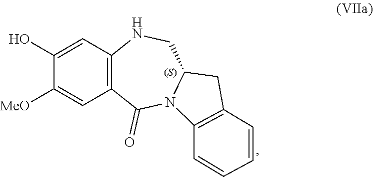
comprising the step of reacting a compound of formula (VIa): 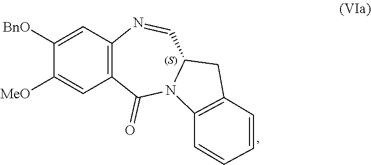
with H₂ in the presence of a Raney nickel catalyst.

38. The method of claim 37, wherein the reaction is carried out in MeOH.

39. The method of claim 38, wherein the reaction is carried out at a temperature between 40° C. and 60° C. or between 45° C. and 55° C.

40. The method of any one claims 37-39, further comprising the step of contacting the reaction mixture with a metal scavenger.

41. The method of any one of claims 37-40, wherein the method further comprises contacting the reaction mixture with a silica-based scavenger.

42. The method of claim 41, wherein the silica-based scavenger is silicathiol.

43. The method of any one of claims 37-42, wherein the compound of formula (VIIa) is purified by crystallization.

44. The method of claim 43, wherein the crystallization is carried out in MeOH and water.

45. A method of preparing a compound of formula (VIIIa): 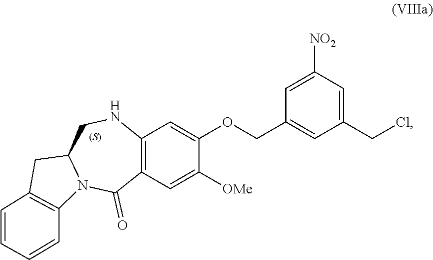
comprising the step of reacting a compound of formula (VIIa): 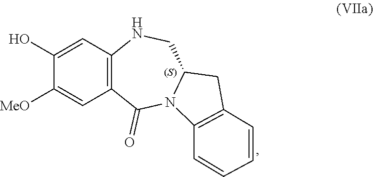
with a compound of formula (c): 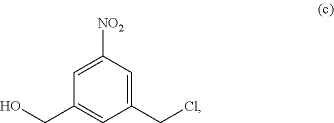 [SMILES: O=[N+]([O-])c1cc(CO)cc(CCl)c1]
to form the compound of formula (VIIIa), wherein the reaction is carried out in the presence of 1,2-bis(diphenylphosphino)ethane (DPPE) and 1,1′-(azodicarbonyl)dipiperidine (ADDP).

46. The method of claim 45, wherein the reaction is carried out in THF.

47. The method of claim 45 or 46, wherein the reaction is carried out at a temperature between 5° C. and 15° C.

48. The method of any one of claims 45-47, wherein the compound of formula (VIIIa) is purified by chromatography.

49. The method of any one of claims 45-47, wherein the compound of formula (VIIIa) is purified by crystallization.

50. The method of claim 49, wherein the compound of formula (VIIIa) is crystallized as an acid addition salt.

51. The method of claim 50, wherein the acid is (+)—O,O′-Di-p-toluoyl-D-tartaric acid (D-(+)-DTTA).

52. The method of any one of claims 45-51, further comprising the step of reacting the compound of formula (VIIIa) with a compound of formula (d): 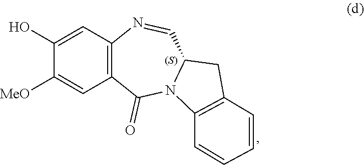
to form a compound of formula (IXa):

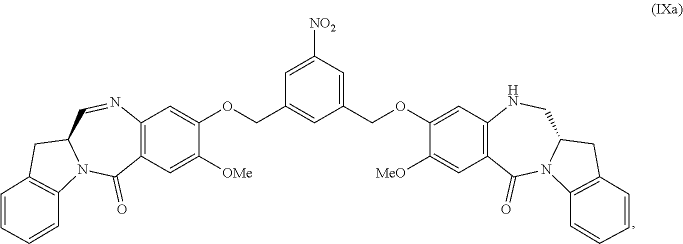 [SMILES: COc1cc2c(cc1OCc1cc(COc3cc4c(cc3OC)C(=O)N3c5ccccc5C[C@H]3CN4)cc([N+](=O)[O-])c1)N=C[C@@H]1Cc3ccccc3N1C2=O]

53. The method of claim 52, wherein the compound of formula (IXa) is purified by precipitation.

54. The method of claim 53, wherein the precipitation is carried out in a mixture of acetonitrile and water.

55. The method of claim 54, wherein the ratio of the acetonitrile and water is 1:1 (v/v)

56. The method of any one of claims 52-55, further comprising the step of reacting the compound of formula (IXa) with a reducing agent to form a compound of formula (IVa):

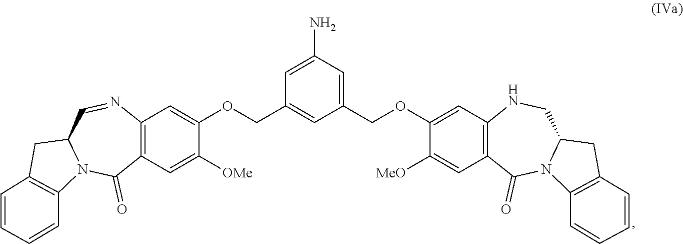 [SMILES: COc1cc2c(cc1OCc1cc(N)cc(COc3cc4c(cc3OC)C(=O)N3c5ccccc5C[C@H]3CN4)c1)N=C[C@@H]1Cc3ccccc3N1C2=O]

57. The method of claim 56, wherein the reducing agent is Fe/NH₄Cl.

58. The method of any one of claims 45-57, wherein the compound of formula (c) is prepared by reacting a compound of formula (c1) 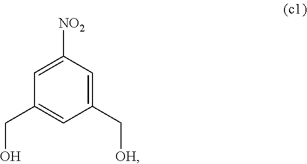 [SMILES: O=[N+]([O-])c1cc(CO)cc(CO)c1]
with hydrochloric acid in toluene.
